(19) United States
(12) Patent Application Publication
(10) Pub. No.: US 2004/0067960 A1
(43) Pub. Date: Apr. 8, 2004

(54) Heterocyclic substituted piperazines for the treatment of schizophrenia

(76) Inventors: Jamie Marie Davis, Ann Arbor, MI (US); Tracy Fay Gregory, Parma, MI (US); Michael Anthony Walters, Novi, MI (US)

Correspondence Address: WARNER-LAMBERT COMPANY, 2800 PLYMOUTH RD, ANN ARBOR, MI, 48105, US

(21) Appl. No.: 10/672,949

(22) Filed: Sep. 26, 2003

Related U.S. Application Data
(60) Provisional application No. 60/413,839, filed on Sep. 26, 2002.

(51) Int. Cl. A61K 31/496; A61K 31/4709; A61K 31/454; C 07D 4 3/02

(52) U.S. Cl. 514/253.05; 514/253.06; 514/253.01; 514/254.02; 514/254.03; 514/254.05; 514/254.06; 514/254.09; 514/255.03; 514/326; 544/360; 544/363; 544/368; 544/373; 544/371; 546/148; 546/169; 514/314; 514/307; 546/197; 546/198

(57) ABSTRACT

This invention relates to compounds of the formula 1

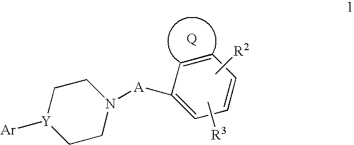

wherein Ar, A, R², R³, Y and ring Q are defined as in the specification, pharmaceutical compositions containing them and their use in the treatment of central nervous system disorders.

### BACKGROUND OF THE INVENTION

This invention relates to heterocyclic substituted piperazines, pharmaceutical compositions containing them and their use for the treatment of schizophrenia and other central nervous system (CNS) disorders.

The heterocyclic substituted piperazine derivatives of this invention exhibit activity as antagonists of dopamine D2 receptors and of serotonin 2A (5HT2A) receptors. They also exhibit partial agonist activity at 5HT1A receptors.

Other heterocyclic piperazine derivatives that are useful for the treatment of schizophrenia are referred to in U.S. Pat. No. 5,350,747, which issued on Sep. 27, 1994, and in U.S. Pat. No. 6,127,357, which issued on Oct. 3, 2000. These patents are incorporated herein by reference in their entireties.

Other piperazine and piperidine derivatives that have been stated to be useful as antipsychotic agents are those referred to in PCT patent publication WO 93/04684, which published on Mar. 18, 1993, and European patent application EP 402644A, which was published on Dec. 19, 1990. These patent applications are incorporated herein by reference in their entireties.

### SUMMARY OF THE INVENTION

The present invention relates to compounds of the formula 1

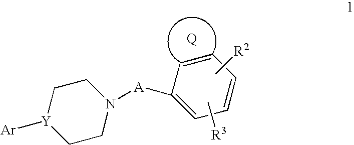

wherein Ar is 1,2-benzisothiazoyl, 1,2-benzisothiazoyl-1-oxide, 1,2-benzisothiazoyl-1-dioxide, 1,2-benzisoxazoyl, naphthyl, pyridyl, quinolyl, isoquinolyl, benzothiadiazolyl, benzotriazolyl, benzoxazolyl, benzoxazolonyl, phthalazinyl, indolyl, indanyl, 1H-indazoyl, or 3-indazolyl, and wherein Ar can optionally be substituted by one or more substituents, preferably from zero to four substituents, independently selected from halo, preferably chloro or fluoro, cyano, nitro, (C₁-C₆) alkyl optionally substituted with from one to three fluorine atoms and (C₁-C₆) alkoxy optionally substituted with from one to three fluorine atoms; with the proviso that Ar can not be attached to the piperazine ring via a phenyl ring of Ar;

Y is N or CH;

A is —(CH₂)ₙCH₂—, wherein n is an integer from one to four, wherein one of the CH₂ groups that is not adjacent to the piperazine nitrogen can optionally be replaced by an oxygen atom;

R² and R³ are independently selected from hydrogen, (C₁-C₆) alkyl optionally substituted with from one to three fluorine atoms, (C₁-C₆) alkoxy optionally substituted with from one to three fluorine atoms, halogen, nitro, cyano, amino, (C₁-C₆) alkylamino and di-(C₁-C₆) alkylamino; and

ring Q can be a saturated, unsaturated or aromatic five to seven membered monocyclic heterocyclic ring containing from one to three heteoratoms independently selected from oxygen, nitrogen and sulfur, and wherein ring Q can be optionally substituted with from one to four substituents, preferably with two or three substituents, independently selected from amino, oxo, hydroxy, (C₁-C₆) alkyl optionally substituted with from one to three fluorine atoms, (C₁-C₆) alkoxy optionally substituted with from one to three fluorine atoms, aryl, aryl-(C₁-C₆) alkyl-, (C₁-C₆) alkenyl optionally substituted with from one to three fluorine atoms, heteroaryl and heteroaryl-(C₁-C₆) alkyl-, wherein the alkyl moieties of the aryl-(C₁-C₆) alkyl- and heteroaryl-(C₁-C₆) alkyl groups can be optionally substituted with from one to three fluoro atoms, and where the aryl and heteroaryl moieties of these groups can optionally be substituted with one or more substituents, preferably from zero to two substutuents, independently selected from halo, oxo, nitro, amino, cyano, (C₁-C₆) alkyl optionally substituted with from one to three fluorine atoms and (C₁-C₆) alkoxy optionally substituted with from one to three fluorine atoms; and wherein one of the substituents on ring Q can be an alkyl chain that forms a 3 to 6 membered spirocyclic ring with a carbon atom of ring Q that is not adjacent to a heteroatom of ring Q; with the proviso that there can not be more than one oxo substituent on ring Q and there can not be more than one spirocyclic alkyl substituent on ring Q;

and the pharmaceutically acceptable salts of such compounds.

A preferred embodiment of this invention relates to compounds of the formula 1A

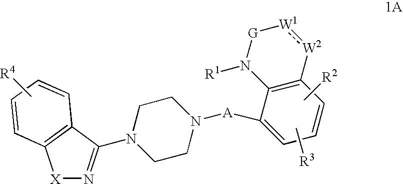

wherein X is sulfur, SO, SO₂, oxygen, or NR;

R is hydrogen, (C₁-C₆) alkyl optionally substituted with from one to three fluorine atoms, (C₁-C₆) alkoxy optionally substituted with from one to three fluorine atoms, aryl, —C(O)(C₁-C₃) alkyl, or —C(O)(C₁-C₃) alkoxy;

A is —(CH₂)ₙCH₂—, wherein n is an integer from one to four, wherein one of the CH₂ groups that is not adjacent to the piperazine nitrogen can optionally be replaced by an oxygen atom;

R¹, R⁵, R⁶, R⁷, R⁸, R⁹ and R¹⁰ are independently selected from hydrogen, (C₁-C₆) alkyl optionally substituted with from one to three fluorine atoms, (C₁-C₆) alkoxy optionally substituted with from one to three fluorine atoms, aryl, aryl-(C₁-C₆) alkyl-, (C₁-C₆) alkenyl optionally substituted with from one to three fluorine atoms, heteroaryl and heteroaryl-(C₁-C₆) alkyl-, wherein the alkyl moieties of the aryl-(C₁-C₆) alkyl- and heteroaryl-(C₁-C₆) alkyl groups can be optionally substituted with from one to three fluoro atoms, and where the aryl and heteroaryl moieties of these groups can optionally be substituted with one or more substituents, preferably from zero to two substituents, independently selected from halo, nitro, amino, cyano, (C₁-C₆) alkyl optionally substituted with from one to three fluorine atoms and (C₁-C₆) alkoxy optionally substituted with from one to three fluorine atoms;

or R¹ is ZR⁹ wherein Z is —C(O)—, —C(O)O—, —C(O)NH—, —S(O)₂— or —S(O)₂NR¹⁰, wherein the hyphen to the left of each of the foregoing moieties represents the bond to NR¹ in structural formula 1A, and the hyphen to the right of each of the foregoing moieties represents the bond to R⁹ in structural formula 1A;

R², R³ and R⁴ are independently selected from hydrogen, (C₁-C₆) alkyl optionally substituted with from one to three fluorine atoms, (C₁-C₆) alkoxy optionally substituted with from one to three fluorine atoms, hydroxy, halogen, nitro, cyano, amino, (C₁-C₆) alkylamino and di-(C₁-C₆) alkylamino;

G is —C(═O)— or CH₂;

W¹ is C(R⁵)(R⁶), CHN(R⁵)(R⁶), CHC(═O)NR⁵R⁶ or C(OH)(R⁵);

W² is C(R⁷)(R⁸), CHN(R⁷)(R⁵), CHC(═O)NR⁵R⁶ or C(OH)(R⁷);

the broken line extending from W¹ to W² represents an optional double bond;

or one of R⁵, R⁶, R⁷, and R⁸, if present, that is attached to a carbon atom, can form, together with the carbon to which it is attached and together with another of R⁵, R⁶, R⁷, and R⁸ that is present and attached to a carbon or nitrogen atom, and the carbon or nitrogen atom to which it is attached, a three to seven membered saturated or unsaturated carbocyclic or heterocyclic ring; and

with the proviso that when the there is a double bond between W¹ and W², then: (a) if W¹ is C(R⁵)(R⁶), either R⁵ or R⁶ is absent; and (b) if W¹ is CHN(R⁵)(R⁶), either the H atom attached to the ring carbon or R⁵ or R⁶ is absent; and (c) if W¹ is C(OH)(R⁵), either the OH group attached to the ring carbon or R⁵ is absent; (d) if W¹ is CHC(═O)NR⁵R⁶, either the hydrogen attached to the ring carbon or C(═O)NR⁵R⁶ is absent; (e) if W² is C(R⁷)(R⁸), either R⁷ or R⁸ is absent; (f) if W² is CHN(R⁷)(R⁸), either the H atom or R⁷ or R⁸ is absent; (g) if W² is C(OH)(R⁷), either the OH group or R⁷ is absent; and (h) if W¹ is CHC(═O)NR⁷R⁸, either the hydrogen attached to the ring carbon or C(═O)NR⁷R⁸ is absent;

and the pharmaceutically acceptable salts of such compounds.

Preferred compounds of the invention include the following compounds and their pharmaceutically acceptable salts:

8-[2-(4-1,2-Benzisothiazol-3-yl-piperazin-1-yl)-ethyl]-4,4-dimethyl-3,4-dihydro-1H-quinolin-2-one;

8-[2-(4-1,2-Benzisothiazol-3-yl-piperazin-1-yl)-ethyl]-1,4,4-trimethyl-3,4-dihydro-1H-quinolin-2-one;

8-[2-(4-1,2-Benzisothiazol-3-yl-piperazin-1-yl)-ethyl]-6-fluoro-4,4-dimethyl-3,4-dihydro-1H-quinolin-2-one;

8-[2-(4-1,2-Benzisothiazol-3-yl-piperazin-1-yl)-ethyl]-3,4-dihydro-1H-quinolin-2-one hydrochloride;

8-[2-(4-1,2-Benzisothiazol-3-yl-piperazin-1-yl)-ethyl]-3,3-dimethyl-3,4-dihydro-1H-quinolin-2-one;

8-[2-(4-1,2-Benzisothiazol-3-yl-piperazin-1-yl)-ethyl]-4-phenyl-3,4-dihydro-1H-quinolin-2-one;

8-[2-(4-1,2-Benzisothiazol-3-yl-piperazin-1-yl)-ethyl]-3-methyl-1H-quinolin-2-one;

8-[2-(4-1,2-Benzisothiazol-3-yl-piperazin-1-yl)-ethyl]-3,4-dimethyl-1H-quinolin-2-one;

8-[2-(4-1,2-Benzisothiazol-3-yl-piperazin-1-yl)-ethyl]-4-methyl-1H-quinolin-2-one;

8-[2-(4-1,2-Benzisothiazol-3-yl-piperazin-1-yl)-ethyl]-4-phenyl-1H-quinolin-2-one;

8-[2-(4-1,2-Benzisothiazol-3-yl-piperazin-1-yl)-ethyl]-4-triflouromethyl-1H-quinolin-2-one;

8-[2-(4-1,2-Benzisothiazol-3-yl-piperazin-1-yl)-ethyl]-4,4,5-trimethyl-3,4-dihydro-1H-quinolin-2-one;

8-[2-(4-1,2-Benzisothiazol-3-yl-piperazin-1-yl)-ethyl]-6-chloro-4,4-dimethyl-3,4-dihydro-1H-quinolin-2-one;

8-[3-(4-1,2-Benzisothiazol-3-yl-piperazin-1-yl)-propyl]-6-chloro-4,4-dimethyl-3,4-dihydro-1H-quinolin-2-one;

8-[2-(4-1,2-Benzisothiazol-3-yl-piperazin-1-yl)-ethyl]-4-isopropyl-1H-quinolin-2-one;

8-[2-(4-1,2-Benzisothiazol-3-yl-piperazin-1-yl)-ethyl]-4-ethyl-1H-quinolin-2-one;

8-[2-(4-1,2-Benzisothiazol-3-yl-piperazin-1-yl)-ethyl]-4-propyl-1H-quinolin-2-one; and

8-[2-(4-1,2-Benzisothiazol-3-yl-piperazin-1-yl)-ethyl]-3-ethyl-4-methyl-1H-quinolin-2-one.

Other preferred embodiments of this invention include compounds of the formula 1A wherein n is one.

Other preferred embodiments of this invention include compounds of the formula 1A wherein R⁴ is hydrogen.

Other preferred embodiments of this invention include compounds of the formula 1A wherein one or both of R² and R³ are hydrogen.

Other preferred embodiments of this invention include compounds of the formula 1A wherein R¹, R⁵, R⁶, R⁷ and R⁸ are selected, independently, from hydrogen and (C₁-C₃)alkyl.

Other embodiments of this invention include the following compounds and their pharmaceutically acceptable salts:

8-[3-(4-1,2-Benzisothiazol-3-yl-piperazin-1-yl)-propyl]-4,4-dimethyl-3,4-dihydro-1H-quinolin-2-one;

8-[3-(4-1,2-Benzisothiazol-3-yl-piperazin-1-yl)-propyl]-4-methyl-3,4-dihydro-1H-quinolin-2-one;

8-[3-(4-1,2-Benzisothiazol-3-yl-piperazin-1-yl)-propyl]-3,3-dimethyl-3,4-dihydro-1H-quinolin-2-one;

8-[2-(4-1,2-Benzisoxazol-3-yl-piperazin-1-yl)-ethyl]-4,4,5-trimethyl-3,4-dihydro-1H-quinolin-2-one;

8-[2-(4-1,2-Benzisoxazol-3-yl-piperazin-1-yl)-ethyl]-6-fluro-4,4-dimethyl-3,4-dihydro-1H-quinolin-2-one;

8-[2-(4-1,2-Benzisoxazol-3-yl-piperazin-1-yl)-ethyl]-4,4,6-trimethyl-3,4-dihydro-1H-quinolin-2-one;

8-{2-[4-(1H-Indazol-3-yl)-piperazin-1-yl]-ethyl}-4,4-dimethyl-3,4-dihydro-1H-quinolin-2-one;

8-{2-[4-(1H-Indazol-3-yl)-piperazin-1-yl]-ethyl}-6-fluoro-4,4-dimethyl-3,4-dihydro-1H-quinolin-2-one;

8-[2-(4-1,2-Benzisoxazol-3-yl-piperazin-1-yl)-ethyl]-4-isopropyl-1H-quinolin-2-one;

8-[2-(4-1,2-Benzisoxazol-3-yl-piperazin-1-yl)-ethyl]-4-ethyl-1H-quinolin-2-one;

8-[2-(4-1,2-Benzisoxazol-3-yl-piperazin-1-yl)-ethyl]-4-propyl-1H-quinolin-2-one;

8-[2-(4-Benzo[d]isothiazol-3-yl-piperazin-1-yl)-ethoxy]-4,4-dimethyl-3,4-dihydro-1H-quinolin-2-one;

8-[2-(4-Benzo[d]isoxazol-3-yl-piperazin-1-yl)-ethoxy]-4,4-dimethyl-3,4-dihydro-1H-quinolin-2-one;

8-[2-(4-Benzo[d]isothiazol-3-yl-piperazin-1-yl)-ethoxy]-6-fluoro-4,4-dimethyl-3,4-dihydro-1H-quinolin-2-one;

8-[2-(4-Benzo[d]isoxazol-3-yl-piperazin-1-yl)-ethoxy]-6-fluoro-4,4-dimethyl-3,4-dihydro-1H-quinolin-2-one;

6-Fluoro-8-{2-[4-(6-fluoro-benzo[d]isoxazol-3-yl)-piperazin-1-yl]-ethoxy}-4,4-dimethyl-3,4-dihydro-1H-quinolin-2-one;

8-{2-[4-(6-Fluoro-benzo[d]isoxazol-3-yl)-piperazin-1-yl]-ethoxy}-4,4-dimethyl-3,4-dihydro-1H-quinolin-2-one;

8-{3-[4-(6-Fluoro-benzo[d]isoxazol-3-yl)-piperazin-1-yl]-propoxy}-4,4-dimethyl-3,4-dihydro-1H-quinolin-2-one;

8-[3-(4-Benzo[d]isoxazol-3-yl-piperazin-1-yl)-propoxy]-6-fluoro-4,4-dimethyl-3,4-dihydro-1H-quinolin-2-one;

8-[3-(4-Benzo[d]isothiazol-3-yl-piperazin-1-yl)-propoxy]-4,4-dimethyl-3,4-dihydro-1H-quinolin-2-one;

8-[3-(4-Benzo[d]isothiazol-3-yl-piperazin-1-yl)-propoxy]-6-fluoro-4,4-dimethyl-3,4-dihydro-1H-quinolin-2-one;

8-[3-(4-Benzo[d]isoxazol-3-yl-piperazin-1-yl)-propoxy]-4,4-dimethyl-3,4-dihydro-1H-quinolin-2-one;

6-Fluoro-8-{3-[4-(6-fluoro-benzo[d]isoxazol-3-yl)-piperazin-1-yl]-propoxy}-4,4-dimethyl-3,4-dihydro-1H-quinolin-2-one;

8-[4-(4-Benzo[d]isoxazol-3-yl-piperazin-1-yl)-butoxy]-4,4-dimethyl-3,4-dihydro-1H-quinolin-2-one;

8-[4-(4-Benzo[d]isothiazol-3-yl-piperazin-1-yl)-butoxy]-4,4-dimethyl-3,4-dihydro-1H-quinolin-2-one;

8-{4-[4-(6-Fluoro-benzo[d]isoxazol-3-yl)-piperazin-1-yl]-butoxy}-4,4-dimethyl-3,4-dihydro-1H-quinolin-2-one;

8-[4-(4-Benzo[d]isoxazol-3-yl-piperazin-1-yl)-butoxy]-6-fluoro-4,4-dimethyl-3,4-dihydro-1H-quinolin-2-one;

8-[4-(4-Benzo[d]isothiazol-3-yl-piperazin-1-yl)-butoxy]-6-fluoro-4,4-dimethyl-3,4-dihydro-1H-quinolin-2-one; and

6-Fluoro-8-{4-[4-(6-fluoro-benzo[d]isoxazol-3-yl)-piperazin-1-yl]-butoxy}-4,4-dimethyl-3,4-dihydro-1H-quinolin-2-one.

Other embodiments of this invention relate to compounds of the formula 1 or 1A wherein the ring that is fused to the R² and R³ containing benzo ring is a six-membered ring.

The term “alkyl”, as used herein, unless otherwise indicated, includes saturated monovalent hydrocarbon radicals having straight, branched or cyclic moieties or combinations thereof. Examples of “alkyl” groups include, but are not limited to, methyl, ethyl, propyl, isopropyl, butyl, iso- sec- and tert-butyl, pentyl, hexyl, heptyl, 3-ethylbutyl, cyclopropyl, cyclobutyl, cyclopentyl, cyclohexyl, cycloheptyl, norbornyl, and the like.

The term “aryl”, as used herein, unless otherwise indicated, includes an aromatic ring system with no heteroatoms (e.g., phenyl or naphthyl).

The term “alkoxy”, as used herein, unless otherwise indicated, means “alkyl-O—”, wherein “alkyl” is as defined above. Examples of “alkoxy” groups include, but are not limited to, methoxy, ethoxy, propoxy, butoxy and pentoxy.

The term “alkenyl”, as used herein, unless otherwise indicated, includes unsaturated hydrocarbon radicals having one or more double bonds connecting two carbon atoms, wherein said hydrocarbon radical may have straight, branched or cyclic moieties or combinations thereof. Examples of “alkenyl” groups include, but are not limited to, -ethenyl, propenyl, butenyl, pentenyl.

The term “heteroaryl” or as used herein, unless otherwise indicated, includes monocyclic aromatic heterocycles containing five or six ring members, of which from 1 to 4 can be heteroatoms selected, independently, from N, S and O, and bicyclic aromatic heterocycles containing from eight to twelve ring members, of which from 1 to 4 can be heteroatoms selected, independently, from N, S and O.

The term “one or more substituents”, as used herein, refers to a number of substituents that equals from one to the maximum number of substituents possible based on the number of available bonding sites.

The terms “halo” and “halogen”, as used herein, unless otherwise indicated, include, fluoro, chloro, bromo and iodo.

The term “treating”, as used herein, refers to reversing, alleviating, inhibiting the progress of, or preventing the disorder or condition to which such term applies, or preventing one or more symptoms of such condition or disorder.

The term “treatment”, as used herein, refers to the act of treating, as “treating” is defined immediately above.

The term “methylene”, as used herein, means —CH₂—.

The term “ethylene”, as used herein, means —CH₂CH₂—.

The term “propylene”, as used herein, means —CH₂CH₂CH₂—.

The compounds of formula 1 and their pharmaceutically acceptable salts are also referred to herein, collectively, as the “novel compounds of this invention” and the “active compounds of this invention”.

This invention also relates to a pharmaceutical composition comprising a therapeutically effective amount of a compound of the formula 1, or a pharmaceutically acceptable salt thereof, and a pharmaceutically acceptable carrier.

Compounds of formula 1 may contain chiral centers and therefore may exist in different enantiomeric and diastereomeric forms. This invention relates to all optical isomers and all stereoisomers of compounds of the formula 1, both as racemic mixtures and as individual enantiomers and diastereoisomers of such compounds, and mixtures thereof, and to all pharmaceutical compositions and methods of treatment defined above that contain or employ them, respectively. Individual isomers can be obtained by known methods, such as optical resolution, optically selective reaction, or chromatographic separation in the preparation of the final product or its intermediate. Individual enantiomers of the compounds of formula 1 may have advantages, as compared with the racemic mixtures of these compounds, in the treatment of various disorders or conditions.

In so far as the compounds of formula 1 of this invention are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the base compounds of this invention are readily prepared by treating the base compound with a substantially equivalent amount of the chosen mineral or organic acid in an aqueous solvent or in a suitable organic solvent, such as methanol or ethanol. Upon careful evaporation of the solvent, the desired solid salt is readily obtained. The acids which are used to prepare the pharmaceutically acceptable acid addition salts of the aforementioned base compounds of this invention are those which form non-toxic acid addition salts, i.e., salts containing pharmaceutically acceptable anions, such as the hydrochloride, hydrobromide, hydroiodide, nitrate, sulfate or bisulfate, phosphate or acid phosphate, acetate, lactate, citrate or acid citrate, tartrate or bi-tartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate))salts.

The present invention also includes isotopically labelled compounds, which are identical to those recited in formula 1, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula 1 of this invention and prodrugs thereof can generally be prepared by carrying out the procedures disclosed in the Schemes and/or in the Examples below, by substituting a readily available isotopically labelled reagent for a non-isotopically labelled reagent.

The compounds of formula 1 of this invention have useful pharmaceutical and medicinal properties.

This invention also relates to a method of treating a disorder or condition selected from the group consisting of single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delerium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to a mammal in need of such treatment an amount of a compound of the formula 1, or a pharmaceutically acceptable salt thereof, that is effective in treating such disorder or condition.

The compounds of formula 1 and their pharmaceutically acceptable salts are also referred to herein, collectively, as the “novel compounds of this invention” and the “active compounds of this invention”.

This invention also relates to a pharmaceutical composition comprising a therapeutically effective amount of a compound of the formula 1, or a pharmaceutically acceptable salt thereof, and a pharmaceutically acceptable carrier.

This invention also relates to a pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delerium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising an amount of a compound of the formula 1, or a pharmaceutically acceptable salt thereof, that is effective in treating such disorder or condition, and a pharmaceutically acceptable carrier.

A more specific embodiment of this invention relates to the above method wherein the disorder or condition that is being treated is selected from major depression, single episode depression, recurrent depression, child abuse induced depression, postpartum depression, dysthymia, cyclothymia and bipolar disorder.

Another more specific embodiment of this invention relates to the above method wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

Another more specific embodiment of this invention relates to the above method wherein the disorder or condition that is being treated is selected from autism, pervasive development disorder, and attention deficit hyperactivity disorder.

Another more specific embodiment of this invention relates to the above method wherein the disorder or condition that is being treated is selected from generalized anxiety disorder, panic disorder, obsessive-compulsive disorder, post-traumatic stress disorder, and phobias, including social phobia, agoraphobia, and specific. phobias.

Another more specific embodiment of this invention relates to the above method wherein the disorder or condition that is being treated is selected from movement disorders such as akinesias, dyskinesias, including familial paroxysmal dyskinesias, spasticities, Tourette's syndrome, Scott syndrome, PALSYS. and akinetic-rigid syndrome; and extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour.

Another more specific embodiment of this invention relates to the above method wherein the disorder or condition that is being treated is selected from delerium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies.

Another more specific embodiment of this invention relates to the above method wherein the compound of formula 1 is administered to a human for the treatment of any two or more comorbid disorders or conditions selected from those disorders and conditions referred to in any of the above methods.

For the treatment of depression, anxiety, schizophrenia or any of the other disorders and conditions referred to above in the descriptions of the methods and pharmaceutical compositions of this invention, the novel compounds of this invention can be used in conjunction with one or more other antidepressants or anti-anxiety agents. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOs), reversible inhibitors of monoamine oxidase (RIMAs), serotonin and noradrenaline reuptake inhibitors (SNRIs), corticotropin releasing factor (CRF) antagonists, α-adrenoreceptor antagonists, and a typical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butripyline, iprindole, lofepramine, nortriptyline, protriptyline, amoxapine, desipramine and maprotiline. Suitable selective serotonin reuptake inhibitors include fluoxetine, fluvoxamine, paroxetine and sertraline. Examples of monoamine oxidase inhibitors include isocarboxazid, pheneizine, and tranylcyclopramine. Suitable reversible inhibitors of monoamine oxidase include moclobemide. Suitable serotonin and noradrenaline reuptake inhibitors of use in the present invention include venlafaxine. Suitable CRF antagonists include those compounds described in International Patent Application Nos. WO 94/13643, WO 94/13644, WO 94/13661, WO 94/13676 and WO 94/13677. Suitable a typical anti-depressants include bupropion, lithium, nefazodone, trazodone and viloxazine. Suitable NK-1 receptor antagonists include those referred to in World Patent Publication WO 01/77100.

Suitable classes of anti-anxiety agents that can be used in combination with the active compounds of this invention include benzodiazepines and serotonin IA (5-HT_{IA}) agonists or antagonists, especially 5-HT_{IA} partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT_{IA} receptor agonists or antagonists include buspirone, flesinoxan, gepirone and ipsapirone.

This invention also relates to a method of treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delerium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example,. due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising administering to said mammal:

(a) a compound of the formula 1 or a pharmaceutically acceptable salt thereof; and

(b) another pharmaceutically active compound that is an antidepressant or anti-anxiety agent, or a pharmaceutically acceptable salt thereof;

wherein the active compounds “a” and “b” are present in amounts that render the combination effective in treating such disorder or condition.

A more specific embodiment of this invention relates to the above method wherein the disorder or condition that is being treated is selected from major depression, single episode depression, recurrent depression, child abuse induced depression, postpartum depression, dysthymia, cyclothymia and bipolar disorder.

Another more specific embodiment of this invention relates to the above method wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

Another more specific embodiment of this invention relates to the above method wherein the disorder or condition that is being treated is selected from autism, pervasive development disorder, and attention deficit hyperactivity disorder.

Another more specific embodiment of this invention relates to the above method wherein the disorder or condition that is being treated is selected from generalized anxiety disorder, panic disorder, obsessive-compulsive disorder, post-traumatic stress disorder, and phobias, including social phobia, agoraphobia, and specific phobias.

Another more specific embodiment of this invention relates to the above method wherein the disorder or condition that is being treated is selected from movement disorders such as akinesias, dyskinesias, including familial paroxysmal dyskinesias, spasticities, Tourette's syndrome, Scott syndrome, PALSYS and akinetic-rigid syndrome; and extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour.

Another more specific embodiment of this invention relates to the above method wherein the disorder or condition that is being treated is selected from delerium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies.

Another more specific embodiment of this invention relates to the above method wherein the compound of formula 1 and the additional antidepressant or anti-anxiety agent are administered to a human for the treatment of any two or more comorbid disorders or conditions selected from those disorders and conditions referred to in any of the above methods.

This invention also relates to a pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delerium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal in need of such treatment, including a human, comprising:

(a) a compound of the formula 1 or a pharmaceutically acceptable salt thereof;

(b) another pharmaceutically active compound that is an antidepressant or anti-anxiety agent, or a pharmaceutically acceptable salt thereof; and

(c) a pharmaceutically acceptable carrier;

wherein the active compounds “a” and “b” are present in amounts that render the composition effective in treating such disorder or condition.

### DETAILED DESCRIPTION OF THE INVENTION

The compounds of formula 1 of the present invention may be prepared as described in the following reaction schemes. Unless otherwise indicated, A, W¹, W², X, Y, R¹ through R¹⁰, ring Q and the dotted line connecting W¹ and W² in the reaction schemes and discussion that follow, are as defined above.

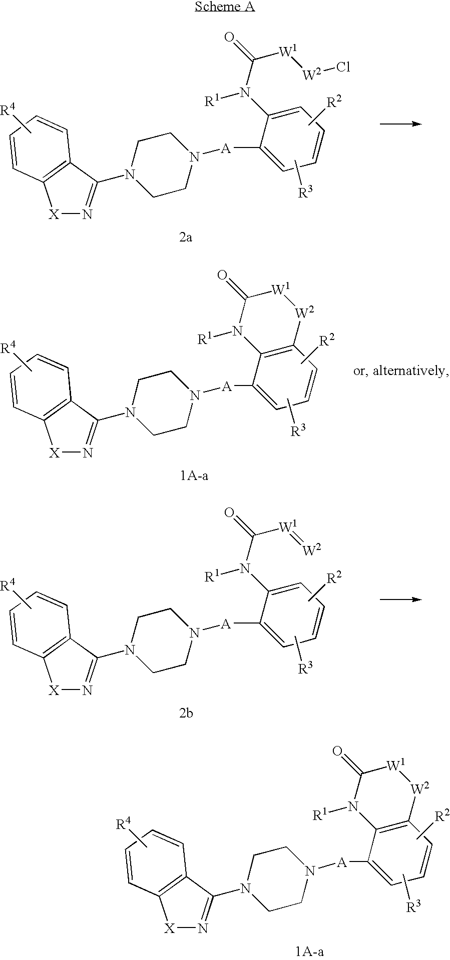

Scheme A illustrates a method for preparing compounds of the formula 1A wherein G is —C(═O)— and W¹ is single bonded to W². These compounds are hereinafter referred to as compounds of the formula 1A-a. This method involves reacting a compound of the formula 2a or 2b with aluminum chloride or another suitable Lewis Acid like aluminum bromide, gallium chloride, iron chloride, zinc chloride, or boron trifluoride. The reaction above may be carried out neat or in any non-polar solvent such as methylene chloride, dichloroethane, benzene, toluene, chlorobenzene, or ortho dichlororbenzene. This reaction is typically carried out at a temperature from about room temperature to about the reflux temperature of the solvent, preferably from about 15° C. to about 180° C., for a period of about 5 minutes to about 48 hours, preferably from about 0.5 to about 16 hours.

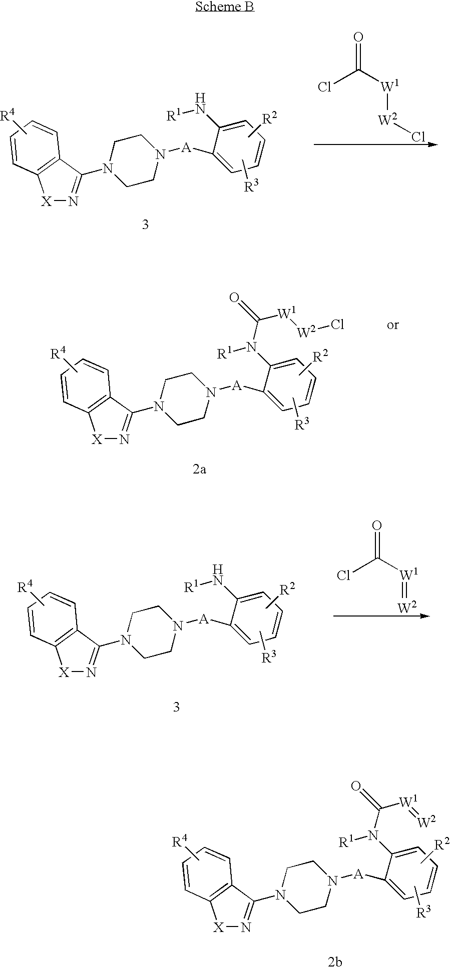

Scheme B illustrates a method for preparing compounds of the formula 2a and 2b by reacting a compound of the formula 3 with a compound of formula W¹W²COCl, wherein either a chlorine substituent can be attached to W² or there can be a double bond between W¹ and W². The reaction above can be carried out in an inert solvent such as methylene chloride, dichloroethane, benzene, toluene, or pyridine. This reaction is typically carried out at a temperature from about −78° C. to about the reflux temperature of the solvent, preferably from about 0° C. to about 25° C., for a period of about 5 minutes to about 48 hours, preferably from about 0.5 to about 16 hours. The reaction is typically performed in the presence of organic base such as diisopropylethylamine, pyridine or triethylamine, preferably triethylamine, or in the presence of a polymer supported base such as resin bound diisopropyl ethyl amine, or resin bound morpholine.

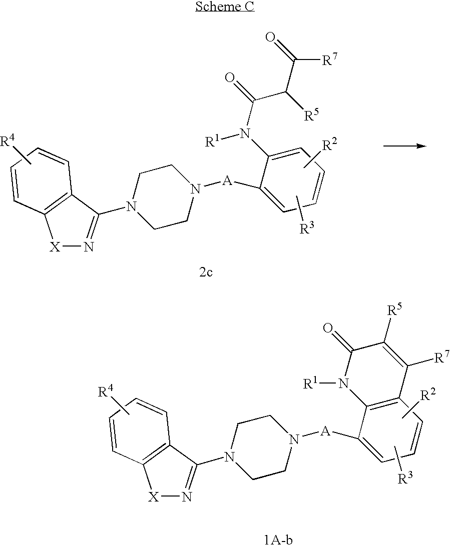

Scheme C illustrates a method for preparing compounds of the formula 1A wherein G is —C(═O)— and W¹ is double bonded to W². These compounds are hereinafter referred to as compounds of the formula 1A-b. This method involves reacting a compound of the formula 2c in sulfuric acid or another suitable acid (e.g. hydrobromic acid, hydroiodic acid or hydrochloric acid). This reaction is typically carried out at a temperature from about room temperature to about the reflux temperature of the solvent, preferably from about 80° C. to about 110° C., for a period of about 10 minutes to about 24 hours, preferably from about 0.5 to about 16 hours.

Scheme D illustrates a method for preparing compounds of the formula 2c by reacting a compound of the formula 3 with a betaketoester of the formula CH₃CH₂OC(O)C(R⁵)C(O)(R⁷).

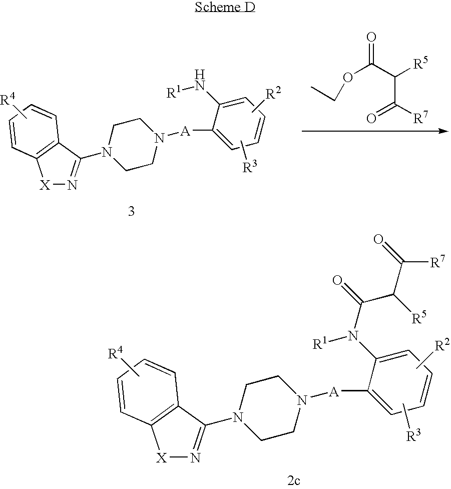

Referring to Scheme D, the reaction above may be carried out neat or in an inert solvent such as xylene, benzene, or toluene. This reaction is typically carried out at a temperature from about 60° C. to about the reflux temperature of the solvent, preferably from about 130° C. to about 160° C., for a period of about 5 minutes to 48 hours, preferably from about 2 to about 5 hours.

Scheme E illustrates a method of preparing compounds of the formula 1 wherein B is —(C═O)— or —(CH₂)—.

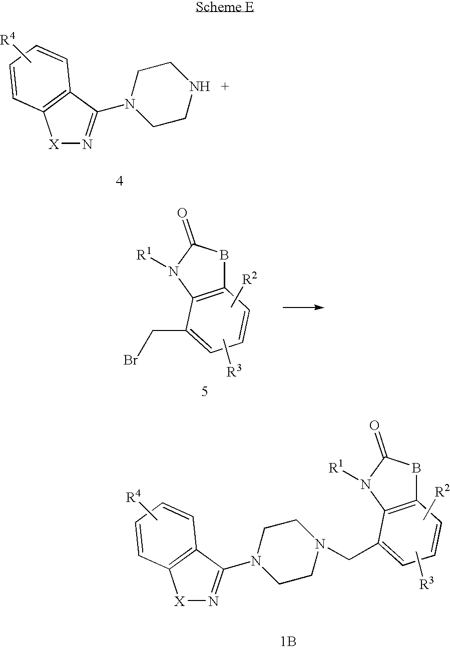

Referring to Scheme E, the halogenated compounds of formula 5 (the bromo substituent can be replaced with fluoro, chloro or iodo) wherein B is —(C═O)— or —CH₂— can be prepared as described by Pavia et al, Benzo-Fused Bicyclic Imides, J. Org. Chem. 1990, 55, 560-564. This reference is incorporated herein by reference in its entirety. The piperazine derivatives of formula 4 can be prepared as described in U.S. Pat. No. 4,831,031, which is referred to above and incorporated herein by reference in its entirety. The coupling of compounds of the formula 4 with compounds of the formula 5 to form the desired compound of formula 1B can also be carried out as described in U.S. Pat. No. 4,831,031. The coupling reaction is generally conducted in a polar solvent such as a lower alcohol, e.g., ethanol, dimethylformamide (DMF) or methylisobutylketone, in the presence of a weak base such as a tertiary amine base, e.g., triethylamine or diisopropylethylamine. Preferably, the reaction is also conducted in the presence of a catalytic amount of sodium iodide and a neutralizing agent for hydrchloride such as sodium or lithium carbonate. The reaction is preferably conducted at the reflux temperature of the solvent used, and can be conducted at a temperature from about 20° C. to about the reflux temperature of the solvent.

Compounds of the formula 1 wherein X is SO or SO₂ can be prepared from the corresponding compounds of the formula 1 wherein X is sulfur using the reaction illustrated in Scheme F. While Scheme F specifically depicts the above transformation for compounds of the formula 1A, the same method can be used to transform all compounds of the formula 1 wherein X is sulfur into the corresponding compounds wherein SO or SO₂.

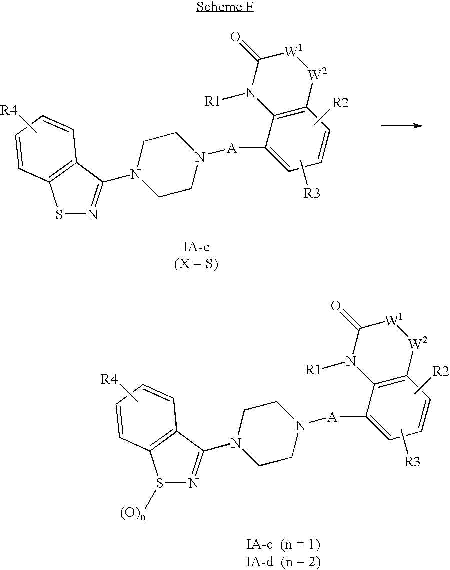

The reaction depicted in Scheme F can be carried out as described by Cipollina, Joseph A. et al. Synthesis and Biological activity of the Putative Metabolites of the A typical Antipsychotic Agent Tiospirone, J. Med. Chem. 1991, 34, 3316-3328. This reaction is typically carried out by heating the compound of formula IA-e with 3-chloroperoxybenzoic acid, 50% hydrogen peroxide, 2-benzenesulfonyl-3-phenyl-oxaziridine or another suitable oxidizing agent. The reaction above may be carried out neat or in a solvent such as methylene chloride, dichloroethane, chloroform, methanol or water. This reaction is typically carried out at a temperature from about −78° C. to about the reflux temperature of the solvent, preferably from about −30° C. to about room temperature, for a period of about 5 minutes to 48 hours, preferably from 0.5 to 16 hours. Compounds of the formulas 1A-c and 1A-d are separated using flash chromatography.

Scheme G illustrates the synthesis of compounds of the formula 1A wherein G is —(C═O)— and A is (CH₂)ₙ—CH₂—O—. Analogous procedures can be used to prepare all compounds of the formula 1 wherein A is (CH₂)ₙ—CH₂—O—.

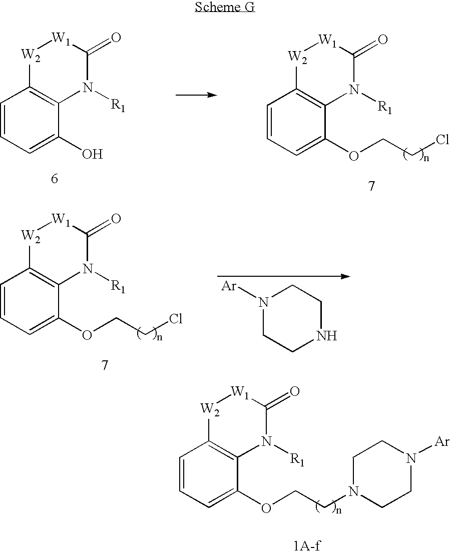

Referring to Scheme G, compounds of the formula 6 can be converted into the corresponding compounds of the formula 7 using the procedure described by Banno et al., Chem. Pharm. Bull, 36, 11; 1988; 4377-4388. Compounds of the formula 7 can be converted into the corresponding compounds of formula 1A4 by the procedure described above for converting compounds of the formula 4 into the corresponding compounds of formula 1 B.

Scheme H illustrates the preparation of compounds of the formula 6.

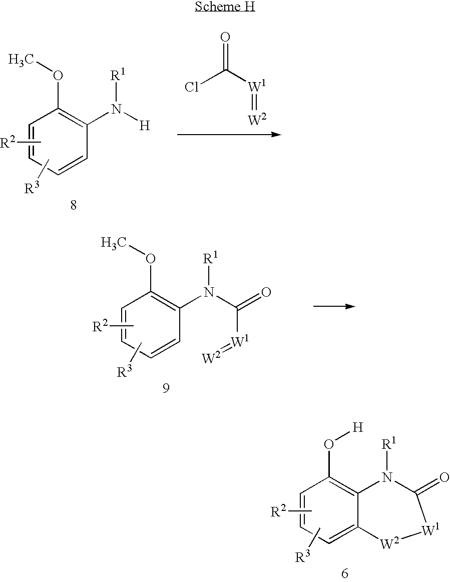

Compounds of formula 6 can be prepared from compounds of formula 8 by applying methods similar to those reported by Shigematsu (Chem. Pharm. Bull. 1961, 9, 970) and Chen, et. al. (J. Chinese Chem. Soc. 2000, 47, 155), and those described above in the preparation of compounds of formula 1A-a from compounds of formula 3.

Compounds of the formula 1A wherein G is CH₂ can be prepared from the corresponding compounds of the formula 1 wherein G is carbonyl using the reaction illustrated in Scheme I. While Scheme I specifically depicts the above transformation for compounds of the formula 1A-g, the same method can be used to transform all compounds of the formula 1 wherein G is carbonyl into the corresponding compounds wherein G is CH₂.

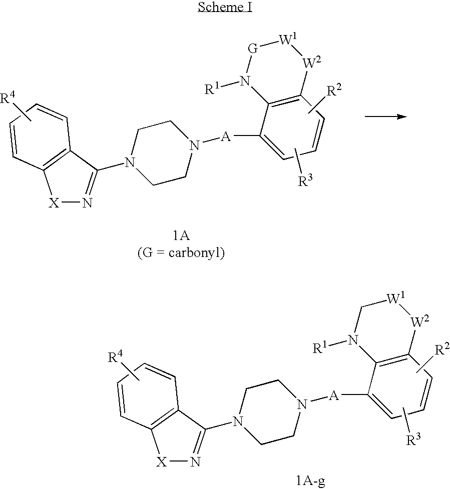

Scheme I illustrates a method for preparing compounds of the formula 1A-g by reducing the amide carbonyl G in a compound of the formula 1A with a reducing agent such as borane THF, or borane dimethyl sulfide. The reaction above can be carried out in a solvent such as methylene chloride, dichloroethane, benzene, or toulene. This reaction is typically carried out at a temperature from about −78° C. to about the reflux temperature of the solvent, preferably from about −20° C. to about 50° C., for a period of about 5 minutes to about 48 hours, preferably from about 0.5 to about 16 hours. The reaction is typically quenched with methanol, water, or a dilute base such as sodium carbonate or sodium bicarbonate. Preferably, the reaction is quenched with methanol or 10% sodium carbonate and the complexes are broken up by heating the reaction mixture to a temperature from about 30° C. to about the reflux temperature of the solvent, preferably to about 90° C., for about 0.5 to about 20 hours, preferably for about 2 hours.

Scheme J illustrates the preparation of Compounds of the formula 1 wherein R¹=Z-R⁹ from the corresponding compounds of the formula 1A-g wherein R¹=H. While Scheme J specifically depicts the above transformation for compounds of the formula 1A-h, the same method can be used to transform all compounds of the formula 1 wherein R¹ is hydrogen into the corresponding compounds wherein R¹ is ZR⁹.

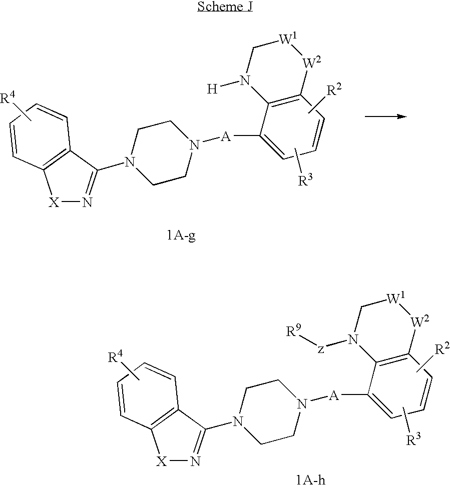

Scheme J illustrates a method for preparing compounds of the formula 1A-h by reacting compounds of the formula 1A-g with a compound of the formula R⁹-T wherein T is —COCl, an acid or a suitably activated acid derivative such as the mixed anhyride, —OCOCl, —N═C═O, or —SO₂Cl, or wherein R⁹-T is ClSO₂N(Me)₂ or ClSO₂R¹⁰. This reaction may be carried out in an inert solvent such as methylene chloride, dichloroethane, benzene, toluene, or pyridine, preferably methylene chloride. Typically, it is carried out at a temperature from about −78° C. to about the reflux temperature of the solvent, preferably from about 0° C. to about 25° C., for a period of about 5 minutes to 48 hours, preferably from about 0.5 to about 16 hours. This reaction is generally performed in the presence of organic base such as diisopropylethylamine, pyridine, or triethylamine, preferably triethylamine, or in the presence of a polymer supported base such as tris-(2-aminoethyl)amine polystyrene.

Compounds of the formula 3 can be prepared as described below in Schemes K through N.

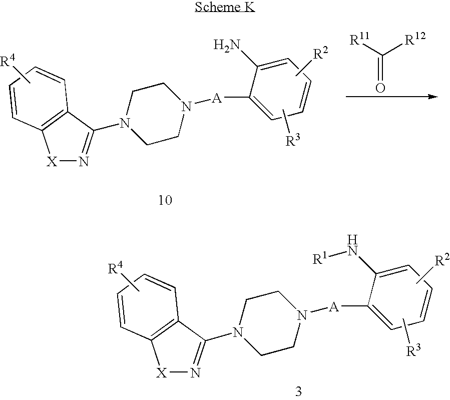

Scheme K illustrates a method for preparing compounds of the formula 3 by the reductive amination of compounds of the formula 10 with compounds of the formula R¹¹R¹²C═O, wherein R¹¹ and R¹² are independently selected from hydrogen, (C₁-C₃) alkyl, aryl, aryl (C₁-C₆) alkyl, (C₁-C₃) alkenyl, heteroaryl, and heteroaryl (C₁-C₆) alkyl, wherein the aryl and heteroaryl moieties of the foregoing R⁵ and R⁶ groups can be optionally substituted with one or two substituents that are independently selected from halo, (C₁-C₆ alkyl) optionally substituted with from one to three fluorine atoms and (C₁-C₆ alkoxy) optionally substituted with from one to three fluorine atoms.

The above reaction may be carried in one vessel without isolation of the imine intermediate, or R¹¹R¹²C═O and the compound of formula 10 may be combined in an inert solvent such as methylene chloride, dichloroethane, toluene or benzene, either at about room temperature or at about the reflux temperature of the solvent, with or without removal of the by product water, to form the imine, which is then reduced. The reduction can be carried out using methods well known to those of skill in the art, for example, by catalytic hydrogenation, or, preferably, with several hydride reagents in a reaction inert solvent. The catalytic hydrogenation can be carried out in the presence of a metal catalyst such as palladium or Raney nickel. Suitable hydride reagents include borohydrides such as sodium borohydride (NaBH₄), sodium cyanoborohydride (NaBH₃CN) and sodium triacetoxyborohydride (NaB(OAc)₃H), boranes, aluminum based reagents and trialkylsilanes. Suitable solvents include polar solvents such as methanol, ethanol, methylene chloride, dichloroethane, tetrahydrofuran (THF), dioxane, toluene, benzene and ethylacetate. This reaction is typically carried out at a temperature from about −78° C. to about the reflux temperature of the solvent, preferably from about 0° C. to about 25° C., for a period of about 5 minutes to about 48 hours, preferably from about 0.5 to 16 hours. The reduction is typically carried out using NaB(OAc)₃H, with or without the addition of acetic acid (HOAc), preferably in a polar solvent like methylene chloride (CH₂Cl₂) or dichloroethane. Alternatively, when R¹¹ and R¹² are hydrogen, the reaction product of formula 2, wherein R² is —CH₃, can be formed by using the method reported in Barluenga, J.; Bayon, A. M.; Asensio, G., JCSCC 1984, 1334-1335.

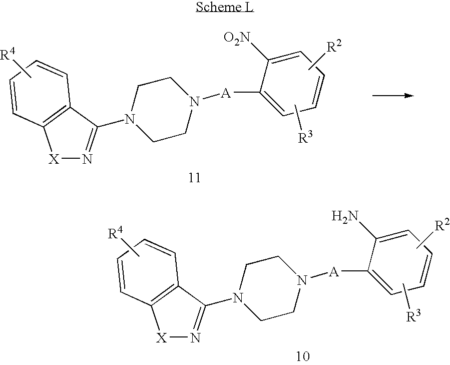

Scheme L illustrates a method for the preparation of compounds of the formula 10 by the reduction of compounds of the formula 11. This reduction can be achieved using standard methodology well known to those of skill in the art, preferably using a Raney nickel catalyst with hydrogen in a solvent such as dimethylformamide (DMF), tetrahydrofuran (THF), 1,4-dioxane, isopropanol, methanol or ethanol, preferably ethanol, in the presence of triethylamine. Other reducing agents that can be employed for this reduction include, but are not limited to, palladium with hydrogen (Pd/H₂) or ammonium formate, tin(II) chloride (SnCl₂), iron/hydrochloric acid (Fe/HCl), iron/acetic acid (Fe/HOAc), or sodium hydrogen sulfide/sodium sulfide (NaSH/NaS₂), in appropriate solvents such as ethyl acetate, DMF, N-methylpyrrolidinone (NMP), methanol, ethanol, isopropanol, dimethylacetamide (DMA), water or THF.

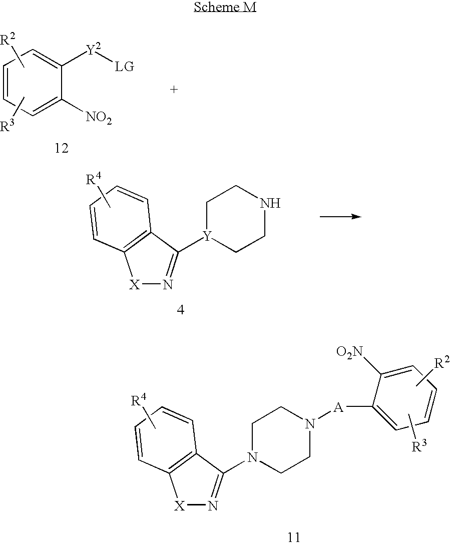

Scheme M illustrates a method for preparing compounds of the formula 11 wherein A is (CH₂)ₙ, n is an integer from one to four and LG is Cl, Br, —OTs (tosylate), or —OMes (mesylate), by the alkylation of compounds of the formula 12 with a readily available piperazine or piperdine of formula 4. This alkylation can be performed in a suitable polar solvent such as DMF, DMSO, ethyl acetate or acetonitrile, preferably acetonitrile, in the presence of a suitable base such as triethylamine or potassium carbonate, preferably K₂CO₃, with or without the addition of a small amount of water and with or without catalytic NaI or KI. The reaction is maintained at a temperature from about 25° C. to about the reflux temperature of the solvent for about 1 to about 24 hours, preferably 15 hours, or heated in a microwave reactor at about 150° C. for about 1-2 hours.

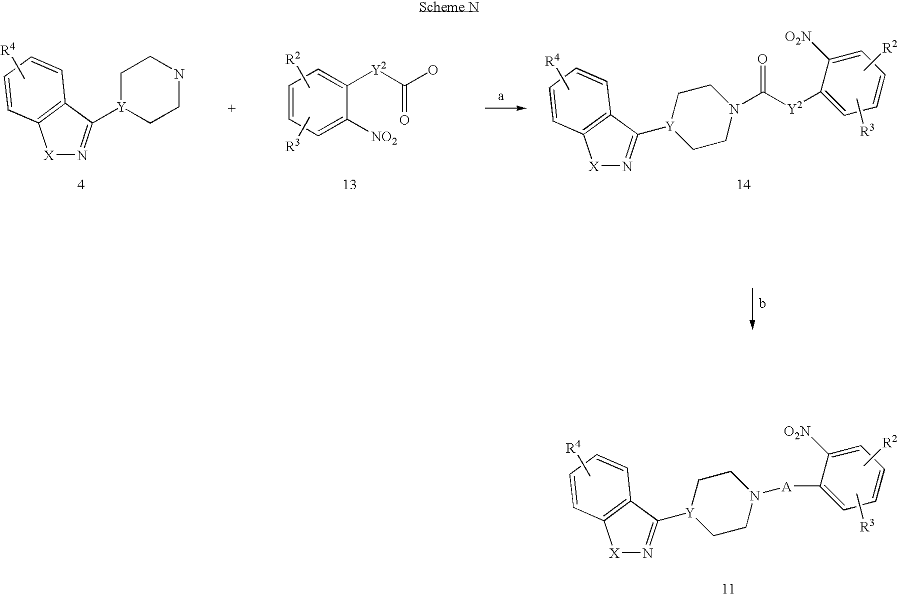

Scheme N illustrates a method for preparing compounds of the formula 11 from the corresponding compounds of the formula 14 wherein Y² is (CH₂)ₙ and n is an integer of from one to three, by amide bond coupling with piperidines or piperazines of the formula 4 followed by reduction of the amide bond in 14. Compound 13 can be made according to the procedures disclosed for similar compounds using appropriate starting materials, see Bull, D. J.; Fray, M. J.; Mackenny, M. C., Malloy, K. A.; Synlett. 1996, 647 and Sun, L.; Tran, N.; Tang, F.; App, H.; Hirth, P.; McMahon, G.; Tang, C.; J. Med. Chem. 1998, 41, 2588-2603. Step A can be accomplished using any standard peptide coupling agent, preferably bis(2-oxo-3-oxazolidinyl)phosphonic chloride (BOP-Cl) at 0° C. to about ambient temperature, for a period of about 1 hour to about 24 hours, in an inert solvent such as dichloroethane or CH₂Cl₂, preferably CH₂Cl₂, to form the corresponding compounds of formula 14. The reduction of compounds of the formula 14 to those of the formula 11 can be performed using many standard reducing agents, preferably using borane dimethylsulfide in toluene at reflux for about 1 hour to about 24 hours.

The preparation of other compounds of the formula 1 and intermediates used in their synthesis that are not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

The compounds of the formula 1, and the intermediates shown in the above reaction schemes can be isolated and purified by conventional procedures, such as recrystallization or chromatographic separation.

The compounds of the formula 1 and their pharmaceutically acceptable salts can be administered to mammals via either the oral, parenteral (such as subcutaneous, intraveneous, intramuscular, intrasternal and infusion techniques), rectal, buccal or intranasal routes. In general, these compounds are most desirably administered in doses ranging from about 3 mg to about 600 mg per day, in single or divided doses (i.e., from 1 to 4 doses per day), although variations will necessarily occur depending upon the species, weight and condition of the patient being treated and the patient's individual response to said medicament, as well as on the type of pharmaceutical formulation chosen and the time period and interval at which such administration is carried out. However, a dosage level that is in the range of about 25 mg to about 100 mg per day is most desirably employed. In some instances, dosage levels below the lower limit of the aforesaid range may be more than adequate, while in other cases still larger doses may be employed without causing any harmful side effects, provided that such higher dose levels are first divided into several small doses for administration throughout the day.

The compounds of the present invention may be administered alone or in combination with pharmaceutically acceptable carriers or diluents by any of the routes previously indicated, and such administration may be carried out in single or multiple doses. More particularly, the novel therapeutic agents of this invention can be administered in a wide variety of different dosage forms, i.e., they may be combined with various pharmaceutically acceptable inert carriers in the form of tablets, capsules, lozenges, troches, hard candies, suppositories, jellies, gels, pastes, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the weight ratio of the novel compounds of this invention to the pharmaceutically acceptable carrier will be in the range from about 1:6 to about 2:1, and preferably from about 1:4 to about 1:1.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intra-articular, intra-muscular and subcutaneous injection purposes. The preparation of all these solutions under sterile conditions is readily accomplished by standard pharmaceutical techniques well known to those skilled in the art.

This invention relates to methods of treating anxiety, depression, schizophrenia and the other disorders referred to in the description of the methods of the present invention, wherein a novel compound of this invention and one or more of the other active agents referred to above (e.g., an NK1 receptor antagonist, tricyclic antidepressant, 5HT1D receptor antagonist, or serotonin reuptake inhibitor) are administered together, as part of the same pharmaceutical composition, as well as to methods in which such active agents are administered separately as part of an appropriate dose regimen designed to obtain the benefits of the combination therapy. The appropriate dose regimen, the amount of each dose of an active agent administered, and the specific intervals between doses of each active agent will depend upon the subject being treated, the specific active agent being administered and the nature and severity of the specific disorder or condition being treated. In general, the novel compounds of this invention, when used as a single active agent or in combination with another active agent, will be administered to an adult human in an amount from about 3 mg to about 300 mg per day, in single or divided doses, preferably from about 25 to about 100 mg per day. Such compounds may be administered on a regimen of up to 6 times per day, preferably 1 to 4 times per day, especially 2 times per day and most especially once daily. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

A proposed daily dose of a 5HT reuptake inhibitor, preferably sertraline, in the combination methods and compositions of this invention, for oral, parenteral or buccal administration to the average adult human for the treatment of the conditions referred to above, is from about 0.1 mg to about 2000 mg, preferably from about 1 mg to about 200 mg of the 5HT reuptake inhibitor per unit dose, which could be administered, for example, 1 to 4 times per day. A proposed daily dose of a 5HT1D receptor antagonist in the combination methods and compositions of this invention, for oral, parenteral, rectal or buccal administration to the average adult human for the treatment of the conditions referred to above, is from about 0.01 mg to about 2000 mg, preferably from about 0.1 mg to about 200 mg of the 5HT1D receptor antagonist per unit dose, which could be administered, for example, 1 to 4 times per day.

For intranasal administration or administration by inhalation, the novel compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch. Formulations of the active compounds of this invention for treatment of the conditions referred to above in the average adult human are preferably arranged so that each metered dose or “puff” of aerosol contains 20 μg to 1000 μg of active compound. The overall daily dose with an aerosol will be within the range 100 μg to 10 mg. Administration may be several times daily, for example 2, 3, 4 or 8 times, giving for example, 1, 2 or 3 doses each time.

All of the title compounds of the examples were tested and at least one stereoisomer of each such compound exhibited a binding affinity for the D2 receptor, measured as percent inhibition at a concentration of 0.1 μm, of no less than 14% and up to 100%. At least one stereoisomer of each such compound exhibited a binding affinity for the 5HT2 receptor, measured as percent inhibition at a concentration of 0.1 μm, of no less than 80% and up to 100%.

The ability of the compounds of this invention to bind to the dopamine D2 and serotonin 2A (5HT2A) receptors can be determined using conventional radioligand receptor binding assays. All receptors can be heterologously expressed in cell lines and experiments conducted in membrane preparations from the cell lines using procedures outlined below. IC₅₀ concentrations can be determined by nonlinear regression of concentration-dependent reduction in specific binding. The Cheng-Prussoff equation can be used to convert the IC₅₀ to Ki concentrations.

Dopamine D2 Receptor Binding:

[³H]Spiperone binding to a membrane preparation from CHO-hD2L cells is carried out in 250 μl of 50 mM Tris-HCl buffer containing 100 mM NaCl, 1 mM MgCl₂ and 1% DMSO at pH 7.4. Duplicate samples containing (in order of addition) the test compounds, 0.4 nM [³H]spiperone and approximately 12 μg protein are incubated for 120 minutes at room temperature. Bound radioligand is separated by rapid filtration under reduced pressure through Whatman GF/B glass fiber filters previously treated with 0.3% polyethyleneimine. Radioactivity retained on the filter is determined by liquid scintillation spectrophotometry.

The title compounds of Examples 1-6 were tested using the above assay, in which specific binding determined in the presence of 1 mM haloperidol was 95%. All of the title compounds of Examples 1-6 exhibited Ki values less than or equal to 1 uM. The title compound of Example 2 exhibited a Ki of 81.32 nM. The title compound of Example 5 exhibited a Ki of 74.87 nM. The title compound of Example 6 exhibited a Ki of 13.82 nM.

Serotonin 2A Binding:

[³H] Ketanserin binding to Swiss-h5HT2A cell membranes can be carried out in 250 μl of 50 mM Tris-HCl buffer pH 7.4. Duplicate samples containing (in order of addition) test compounds, 1.0 nM [³H]ketanserin, and approximately 75 μg protein are incubated for 120 minutes at room temperature. Bound radioligand is separated by rapid filtration under reduced pressure through Whatman GF/B glass fiber filters previously treated with 0.3% polyethyleneimine. Radioactivity retained on the filter is determined by liquid scintillation spectrophotometry.

The title compounds of Examples 1-6 were tested using the above assay, in which specific binding determined in the presence of 1 mM ketanserin was 90%. All of the title compounds of Examples 1-6 exhibited Ki values less than or equal to 1 uM. The title compound of Example 5 exhibited a Ki of 2.07 nM. The title compound of Example 2 exhibited a Ki of 0.18 nM. The title compound of Example 6 exhibited a Ki of 0.04 nM.

d-Amphetamine-Stimulated Locomotor Activity (LMA):

The LMA model is used to test novel compounds for efficacy as orally active dopaminergic (DA) antagonists. In this model, administration of d-amphetamine to rats induces a stimulation of locomotor activity, measured as centimeters traveled over a two-hour period. Compounds are administered prior to d-amphetamine, and their efficacy in decreasing the stimulated locomotion is assessed as a measure of DA antagonism.

(i) Test Animals

Sprague Dawley (S-D) male rats were obtained from Harlan Laboratories, Indianapolis Ind. All rats weighed 130-150 g at the time of arrival and were housed in groups of 5 for at least 1 week prior to testing. Food and water were available ad lib. At the time of testing, rats weighed 150-200 g. Tests occurred between 9:00 AM and 4:00 PM. All rats were food deprived overnight prior to testing.

(ii) Test Apparatus:

Locomotor activity testing in rats was performed using 16-Beam Digiscan Animal Activity Monitors (Accuscan Electronics, Columbus, Ohio). Each test chamber consisted of a Plexiglas box measuring 16×16 inches, placed within the monitor frame. The entire monitor/chamber assembly is further housed inside a stainless steel sound-attenuating chamber (SAC). The SAC is lighted, ventilated, and isolates the rat from room environment. Rats were tested one per chamber. Data is collected using Versamax software.

(iii) Procedure:

Each test consists of four treatment groups, vehicle and three doses of the test compound. Each treatment group is comprised of 8 animals. The test is performed in two sessions, with 4 groups of 4 rats in each treatment group tested in each session, and data from the two sessions, typically morning and afternoon of the same day, combined to give a total of 8 rats per group.

Rats were removed from the housing room and transported to the test room in transfer cages. Each rat was weighed, injected orally via gavage tube with vehicle or one of 3 doses of the test compound. The rat was then placed into an activity monitor, and the door of the SAC closed. After a 30 minute period to allow for drug absorption, each rat was injected subcutaneously with d-amphetamine, 1 mg/kg, replaced into the test chamber, and the monitor turned on. The SAC door was closed, and data collected for 2 hours. At the end of 2 hours, the monitor is switched off, the rats were removed and euthanized.

All injections were administered in a volume of 5 mukg. Test compounds were dissolved or suspended for injection in water containing 0.5% methylcellulose, 1% 1N HCl, and 1% cremaphor EL. d-Amphetamine was dissolved in saline.

(iv) Data Analysis:

Data were collected as centimeters traveled during the 2 hour test period. The effect of the test compound was expressed as a percentage decrease (or increase) in stimulated locomotor activity relative to the activity observed in the vehicle-treated group. Statistical analysis of the data was performed using a one-way analysis of variance (ANOVA), followed by a post-hoc Dunnett's test for each group vs. the vehicle-treated group.

The results are reported as the dose tested in milligrams of test compound per kilogram of test animal (mg/kg). A compound was considered active if it produced a significant decrease in amphetamine-stimulated locomotor activity compared to animals treated with vehicle and amphetamine with compounds typically tested at doses between 0.1 and 10 mg/kg. Minimally effective dose (MED) for reducing d-Amphetamine-stimulated locomotor activity was reported as the lowest dose tested that produced a statistically significant reduction in distance traveled compared to vehicle controls.

The title compounds of Examples 1, 3, 29 were determined to be active in the above assay.

Catalepsy (CAT):

The catalepsy test (CAT) is used as a screen for the propensity of novel compounds to produce extrapyramidal motor side effects (EPS).

When placed in an unusual position, untreated rats will return to a normal position quickly upon being released. Treatment with neuroleptic compounds can increase the amount of time spent in the imposed position.

(i) Test Animals

Sprague Dawley (S-D) male rats were obtained from Harlan Laboratories, Indianapolis, Ind. All rats weighed 130-150 g at the time of arrival and were housed in groups of 6 for 1 week prior to testing. Food and water were available ad lib. At the time of testing, rats weighed 150-200 g. Tests occurred between 8:00 AM and 2:00 PM. All rats were food deprived overnight prior to testing. Eight animals were randomly assigned to groups receiving either vehicle or drug treatment.

(ii) Test Apparatus:

The testing apparatus consisted of a horizontal bar 13 mm in diameter suspended 12 cm from the countertop.

(iii) Procedure:

Rats were brought into the test room in their home cages, weighed and housed individually in a hanging wire rack. Rats were allowed to habituate to the test room for one hour prior to oral administration (PO) of the invention compound or vehicle. Dose ranges used in the CAT test were typically 10 and 30 times the minimally effective dose (MED) in the amphetamine-stimulated locomotor activity test. Two and three hours after dosing rats were individually placed with their forepaws on the horizontal bar and hind limbs on the counter. The amount of time spent in this position was recorded. If a rat remained on the bar less than 26 seconds it received another trial, with up to 3 trials given at each time point. The maximum duration a rat was allowed to remain at the bar was 90 seconds, after which it was returned to the housing rack. At the end of the testing period, rats were sacrificed by carbon dioxide asphyxiation.

(iv) Data Analysis:

Time spent standing at the bar was recorded in seconds. The longest recorded time of the three trials at each of the time points was used in the data analysis with data from the 2 and 3-hour time points analyzed separately. MED for producing catalepsy was reported as the lowest dose of compound (mg/kg) that produces a group mean time on the bar greater than 20 seconds with the majority of rats in that group meeting this criterion. None of the compounds of this invention that were tested produced an MED below 30 mg/kg.

The following Examples illustrate the preparation of the compounds of the present invention. Melting points are uncorrected. NMR data are reported in parts per million and are referenced to the deuterium lock signal from the sample solvent.

### EXAMPLES

Example 1

### 8-[2-(4-1,2-BENZISOTHIAZOL-3-YL-PIPERAZIN-1-YL)-ETHYL]-4,4-DIMETHYL-3,4-DIHYDRO-1H-QUINOLIN-2-ONE

3-Methyl-but-2-enoic acid {2-[2-(4-1,2-benzisothiazol-3-yl-piperazin-1-yl)-ethyl]-phenyl}-amide (200 mg, 0.47 mmol) was dissolved in 4 ml chlorobenzene and aluminum chloride (352 mg, 2.86 mmol) was added at 0° C. The reaction mixture was slowly warmed to 120° C. and stirred for 72 hours (h). The mixture was cooled and the organic layer was removed, washed with water and concentrated. The residue was treated with methanolic hydrochloric acid (HCl) and heated until the solid dissolved. The solution was concentrated, then triturated in hot isopropyl alcohol. 8-[2-(4-1,2-Benzisothiazol-3-yl-piperazin-1-yl)-ethyl]-4,4-dimethyl-3,4-dihydro-1H-quinolin-2-one hydrochloride (17 mg) was isolated in 100% purity @ 254 nm as the hydrochloride salt. LC/MS (APCI): 421 [M+H]⁺; mp 284° C.; ¹H NMR (400 MHz, DMSO-D₆) δ 9.60 (s, 1H), 8.10 (m, 2H), 7.55 (t, J=7.6 Hz, 1H), 7.43 (t, J=7.6 Hz, 1H), 7.20 (d, J=7.6 Hz, 1H), 7.06 (d, J=7.1 Hz, 1H), 6.97 (t, J=7.6 Hz, 1H), 4.08 (d, J=13.4 Hz, 2H), 3.65 (d, J=11.5 Hz, 2H), 3.43 (t, J=12.3 Hz, 2H), 3.29 (m, 4H), 3.06 (m, 2H), 2.31 (s, 2H), 1.18 (s, 6H).

### Example 2

### 8-[2-(4-1,2-BENZISOTHIAZOL-3-YL-PIPERAZIN-1-YL)-ETHYL]-1,4,4-TRIMETHYL-3,4-DIHYDRO-1H-QUINOLIN-2-ONE

Starting from 3-methyl-but-2-enoic acid {2-[2-(4-1,2-benzisothiazol-3-yl-piperazin-1-yl)-ethyl]-phenyl}-methyl-amide (396 mg, 0.91 mmol) and following the procedure as outlined in Example 1, 173 mg of 8-[2-(4-1,2-benzisothiazol-3-yl-piperazin-1-yl)-ethyl]-1,4,4-trimethyl-3,4-dihydro-1H-quinolin-2-one was isolated as an off white powder in 100% purity @ 254 nm; LC/MS (APCI): 435 [M+H]⁺; mp 261° C. ¹HNMR (400 MHz, CDCl₃) δ 7.84 (m, 2H), 7.52 (t, J=7.4 Hz, 1H), 7.40 (t, J=7.4 Hz, 1H), 7.22 (m, 2H), 7.13 (m, 1H), 4.14 (m, 4H), 3.52 (m, 4H), 3.39 (s, 3H), 3.13 (m, 4H), 2.41 (s, 2H), 1.25 (s, 6H).

### Example 3

### 8-[2-(4-1,2-BENZISOTHIAZOL-3-YL-PIPERAZIN-1-YL)- ETHYL]-6-FLUORO-4,4-DIMETHYL-3,4-DIHYDRO-1H-QUINOLIN-2-ONE

Starting from 3-methyl-but-2-enoic acid {2-[2-(4-1,2-benzisothiazol-3-yl-piperazin-1-yl)-ethyl]-4-fluoro-phenyl}-amide (1.73 g, 3.95 mmol) and following the procedure as outlined in Example 1, 670 mg of 8-[2-(4-1,2-benzisothiazol-3-yl-piperazin-1-yl)-ethyl]-6-fluoro-4,4-dimethyl-3,4-dihydro-1H-quinolin-2-one hydrochloride was isolated as an off white powder in 100% purity @ 254 nm; LC/MS (APCI): 439 [M+H]⁺; mp 298° C. ¹HNMR (400 MHz, DMSO-D₆) δ 8.08 (m, 2H), 7.55 (t, J=7.6 Hz, 1H), 7.43 (t, J=7.6 Hz, 1H), 7.06 (dd, J=9.5, 2.6 Hz, 1H), 6.96 (dd, J=9.5, 2.6 Hz, 1H), 4.08 (d, J=13.4 Hz, 2H), 3.65 (d, J=11.7 Hz, 2H), 3.44 (t, J=12.3 Hz, 2H), 3.30 (m, 4H), 3.08 (m, 2H), 2.31 (s, 2H), 1.18 (s, 6H).

### Preparation 1

### {2-[2-(4-1,2-Benzisothiazol-3-yl-piperazin-1-yl)-ethyl]-phenyl}-3-chloro-propionamide

2-[2-(4-1,2-Benzisothiazol-3-yl-piperazin-1-yl)-ethyl]-phenylamine (1.5 g, 4.43 mmol) was dissolved in 100 mL tetrahydrofuran (THF) and triethylamine was added (0.62 mL, 4.43 mmol). 3-Chloropropionyl chloride (0.45 mL, 4.66 mmol) was added under stirring and the reaction stirred at 0° C. for 45 min. The reaction mixture was concentrated under nitrogen and dissolved in 150 mL methylene chloride and then washed with water. The organic layer was concentrated and evaluated by LCMS. The mixture was purified by MPLC (medium pressure liquid chromatography) using a Biotage 40s prepacked silica gel cartridge eluting with 3% methanol in methylene chloride. {2-[2-(4-1,2-benzisothiazol-3-yl-piperazin-1-yl)-ethyl]-phenyl}-3-chloro-propionamide (0.83 g) was isolated in 100% purity @ 254 nm; LC/MS (APCI): 429 [M+H]⁺.

### Example 4

### 8-[2-(4-1,2-BENZISOTHIAZOL-3-YL-PIPERAZIN-1-YL)-ETHYL]-3,4-DIHYDRO-1H-QUINOLIN-2-ONE HYDROCHLORIDE

Starting from {2-[2-(4-1,2-benzisothiazol-3-yl-piperazin-1-yl)-ethyl]-phenyl}-3-chloro-propionamide (0.83 g, 1.94 mmol) and following the procedure as outlined in Example 1, 270 mg of 8-[2-(4-1,2-benzisothiazol-3-yl-piperazin-1-yl)-ethyl]-3,4-dihydro-1H-quinolin-2-one hydrochloride was isolated as a tan solid in 100% purity @ 254 nm; LCMS (APCI): 393 [M+H]⁺; mp 251° C. ¹HNMR (400 MHz, DMSO-D₆) δ 8.08 (m, 2H), 7.55 (t, 1H), 7.43 (t, 1H), 7.06 (m, 1H), 6.96 (m, 2H), 4.08 (d, J=13.4 Hz, 2H), 3.73 (m, 4H), 3.31 (m, 8H), 3.05 (m, 2H).

### Preparation 2

### {2-[2-(4-1,2-Benzisothiazol-3-yl-piperazin-1-yl)-ethyl]-phenyl}-3-chloro-2,2-dimethyl-propionamide

Starting from 2-[2-(4-1,2-benzisothiazol-3-yl-piperazin-1-yl)-ethyl]-phenylamine (338 mg, 1 mmol) and 3-chloropivaloyl chloride and following the procedure as outlined in Preparation 1, 442 mg of {2-[2-(4-1,2-benzisothiazol-3-yl-piperazin-1-yl)-ethyl]-phenyl}-3-chloro-2,2-dimethyl-propionamide was isolated as a white solid. MS (APCI): 457 [M+H]⁺.

### Example 5

### 8-[2-(4-1,2-BENZISOTHIAZOL-3-YL-PIPERAZIN-1-YL)-ETHYL]-3,3-DIMETHYL-3,4-DIHYDRO-1H-QUINOLIN-2-ONE

Starting from {2-[2-(4-1,2-benzisothiazol-3-yl-piperazin-1-yl)-ethyl]-phenyl}-3-chloro-2,2-dimethyl-propionamide (0.20 g, 0.44 mmol) and following the procedure as outlined in Example 1, 15 mg of 8-[2-(4-1,2-Benzisothiazol-3-yl-piperazin-1-yl)-ethyl]-3,3-dimethyl-3,4-dihydro-1H-quinolin-2-one was isolated as an off white solid in 100% purity @ 254 nm; LCMS (APCI): 421 [M+H]⁺.; mp 95° C. ¹HNMR (400 MHz, DMSO-D₆) δ 8.08 (m, 2H), 7.51 (m, 1H), 7.38 (m, 1H), 6.97 (m, 2H), 6.83 (m, 1H), 3.49 (m, 4H), 2.76-2.44 (band, 10H), 0.97 (m, 6H).

### Preparation 3

### N-[2-[2-(4-Benzordlisothiazol-3-yl-piperazin-1-yl)-ethyl]-Phenyl]-3-phenyl-acrylamide

Starting from 2-[2-(4-1,2-benzisothiazol-3-yl-piperazin-1-yl)-ethyl]-phenylamine (5 g, 14.8 mmol) and cinnamoyl chloride (2.58 g, 15.5 mmol) and following the procedure as outlined in Preparation 1, 6.8 g of N-{2-[2-(4-benzo[d]isothiazol-3-yl-piperazin-1-yl)-ethyl]-phenyl}-3-phenyl-acrylamide was isolated as a white powder. LCMS (APCI): 469 [M+H]⁺.

### Example 6

### 8-[2-(4-1.2-BENZISOTHIAZOL-3-YL-PIPERAZIN-1-YL)-ETHYL]-4-PHENYL-3,4-DIHYDRO-1H-QUINOLIN-2-ONE

Starting from N -{2-[2-(4-benzo[d]isothiazol-3-yl-piperazin-1-yl)-ethyl]-phenyl}-3-phenyl-acrylamide (6.7 g, 14.3 mmol) and following the procedure as outlined in Example 1, 1.53 g of 8-[2-(4-1,2-benzisothiazol-3-yl-piperazin-1-yl)-ethyl]-4-phenyl-3,4-dihydro-1H-quinolin-2-one was isolated as a white powder in 100% purity @ 254 nm; LC/MS (APCI): 469 [M+H]⁺; mp 227° C. ¹HNMR (400 MHz, DMSO-D₆) δ 8.12 (m, 2H), 7.64 (m, 3H), 7.42 (m, 2H), 7.26 (m, 2H), 7.15 (m, 2H), 6.92 (m, 1H), 4.10 (m, 2H), 3.65 (m, 2H), 3.45 (m, 8H), 3.13 (m, 2H).

### Preparation 4

### 2-Nitrophenethyl tosylate

2-Nitrophenethyl alcohol (15 g, 89.7 mmol) was dissolved in 450 mL methylene chloride. Triethylamine (37.5 mL, 269 mmol) was added over 10 min and the reaction mixture was stirred at 0° C. for 1 hour (h). Tosyl chloride (20.52 g, 110 mmol) was added slowly to the mixture at 0° C. The reaction was stirred at room temperature (rt) overnight and was concentrated. The residue was dissolved in methylene chloride and washed with water, 1 N hydrochloric acid (HCl), then water. The organic layer was dried over sodium sulfate and evaporated. The residue was triturated with hexanes and 26.44 grams (g) of off-white crystals were collected. Yield 92%; ¹H NMR (400 MHz, CDCl₃) δ 7.93 (d, J=9.7 Hz, 1H), 7.91 (d, J=9.7 Hz, 2H), 7.64 (t, 1H), 7.39 (m, 2H), 7.24 (s, 2H), 4.32 (t, J=6 Hz, 2H), 3.24 (t, J=6 Hz, 2H), 2.41 (s, 3H).

### Preparation 5

### 3-{4-[2-(2-Nitro-phenyl)-ethyl]-piperazin-1-yl}-1,2-benzisothiazole

Excess dried, -325 mesh potassium carbonate (20 g) was diluted in 500 mL acetone and 3-piperazin-1-yl-benzoisothiazole hydrochloride (13.37 g, 52.4 mmol was added. The mixture was stirred for 15 min before 2-nitrophenethyl tosylate (15.3 g, 47.7 mmol) and catalytic 18-crown-6 (0.5 g, 1.9 mmol) was added. The mixture was stirred at reflux for 42 h. After cooling, the salts were filtered off and washed with acetone and the filtrate was concentrated. The residue was taken up in methylene chloride and washed with water. The organic layer was dried over sodium sulfate, and concentrated. The residue was triturated with ethyl acetate and the collected solid was washed with ethyl ether and dried in vacuo to afford 13.08 g of a viscous, brown liquid. Yield 75%; MS (APCI): 369 [M+H]⁺.

### Preparation 6

### 2-[2-(4-1,2-Benzisothiazol-3-yl-piperazin-1-yl)-ethyl]-phenylamine

3-{4-[2-(2-Nitro-phenyl)-ethyl]-piperazin-1-yl}-1,2-benzisothiazole (12.93 g, 35.13 mmol) was dissolved in 150 mL of THF treated with triethylamine (5 mL) and wet Raney nickel (3 g). The resulting mixture was placed on a shaker type hydrogenator, purged with hydrogen, pressurized (two re-pressurizations were needed to maintain the pressure between 3 and 17 psig) and shaken at room temperature for 64 h. The resulting mixture was filtered to remove the catalyst then filtered a second time over celite before the filtrate was concentrated. The resultant white solid was triturated with ethyl ether and dried in vacuo (7.88 g). Yield 66%; mp 149° C.; MS (APCI): 339 [M+H]⁺.

### Preparation 7

### (5-Fluoro-2-nitro-phenyl)-acetic Acid

3-Fluoro phenyl acetic acid (5 g, 36.7 mmol) was diluted in 30 mL of chloroform and ammonium nitrate (3.12 g, 38.9 mmol) was added. The reaction mixture was cooled to 0° C. and trifluoro acetic acid anhydride (16.02 mL, 113 mmol) was added dropwise. The reaction stirred at 0° C. for 3 h before water was added to slowly quench the reaction. The chloroform layer was washed with water, collected and dried over Na₂SO₄, and concentrated. The desired isomer crystallized out of the crude solution in ethyl acetate and was then triturated with acetonitrile to afford 5.25 g of the desired isomer as a brown solid. Yield 87%; MS (APCI): 199 [M−H]⁺.

### Preparation 8

### 1-(4-1,2-Benzisothiazol-3-yl-piperazin-1-yl)-2-(5-fluoro-2-nitro-phenyl)-ethanone

3-Piperazin-1-yl-benzoisothiazole hydrochloride (1.31 g, 5.1 mmol) and (5-fluoro-2-nitro-phenyl)-acetic acid (800 mg, 4.3 mmol) were combined in 100 mL methylene chloride with triethylamine (1.20 mL, 8.6 mmol). This solution stirred for 15 min before bis-(2-oxo-3-oxazolidinyl) phosphinic chloride 1.09 g, 4.3 mmol) was added. After stirring overnight at room temperature (rt), the reaction was quenched with water and extracted into methylene chloride. The organic layer was washed with 0.5 N HCl, water, sodium bicarbonate then water before it was dried over sodium sulfate (Na₂SO₄) and concentrated. The organic layer was dried over sodium sulfate, concentrated, and purified by MPLC using a Biotage prepacked silica gel cartridge eluting with 3% methanol in methylene chloride (CH₂Cl₂) to afford 870 mg of an off-white foam. Yield 50%; mp 72° C.; MS (APCI): 401 [M+H]⁺.

### Preparation 9

### 3-{4-[2-(5-Fluoro-2-nitro-phenyl)-ethyl]-piperazin-1-yl}-1,2-benzisothiazole

1-(4-1,2-benzisothiazol-3-yl-piperazin-1-yl)-2-(5-fluoro-2-nitro-phenyl)-ethanone (870 mg, 2.18 mmol) was diluted in 50 mL of toluene. Borane methyl sulfide complex (2.0 M in tolulene, 7.22 mL) was slowly added to the stirring mixture. The reaction mixture was heated to 110° C. in an oil bath overnight. Upon cooling, excess sodium bicarbonate was added dropwise and the mixture was heated to 85° C. until gas evolution subsided. The water layer was removed and extracted in methylene chloride. The organic layers were combined, dried over sodium sulfate (Na₂SO₄), then concentrated and purified via column chromatography and recrystallized in isopropyl alcohol to afford 411 mg of yellow crystals. Yield 49%; mp 131° C.; MS (APCI): 387 [M+H]⁺.

### Preparation 10

### 2-[2-(4-1,2-Benzisothiazol-3-yl-piperazin-1-yl)-ethyl]-4-fluoro-phenylamine

2-[2-(4-1,2-Benzisothiazol-3-yl-piperazin-1-yl)-ethyl]-4-fluoro-phenylamine was prepared according to the general method as outlined in Preparation 6 starting from 3-{4-[2-(5-fluoro-2-nitro-phenyl)-ethyl]-piperazin-1-yl}-1,2-benzisothiazole (2.27 g, 4.7 mmol). The product was isolated via column chromatography and recrystallized in isopropyl alcohol to afford 555 mg off white crystals. Yield 51%; mp 115° C.; MS (APCI): 357 [M+H]⁺.

### Preparation 11

### 3-Methyl-but-2-enoic acid {2-[2-(4-1,2-benzisothiazol-3-yl-piperazin-1-yl)-ethyl]-4-fluoro-phenyl}-amide

Starting from 2-[2-(4-1,2-benzisothiazol-3-yl-piperazin-1-yl)-ethyl]-4-fluoro-phenylamine (300 mg, 0.84 mmol) and 3,3 dimethyl acryloyl chloride (98 μL, 0.88 mmol) and following the procedure as outlined in Preparation 4, 287 mg of 3-methyl-but-2-enoic acid {2-[2-(4-1,2-benzisothiazol-3-yl-piperazin-1-yl)-ethyl]-4-fluoro-phenyl}-amide was isolated as a white powder in 100% purity @ 254 nm; LCMS (APCI): 439 [M+H]⁺; mp 175° C.

### Example 3

### 8-[2-(4-1,2-BENZISOTHIAZOL-3-YL-PIPERAZIN-1-YL)-ETHYL]-6-FLUORO-4,4-DIMETHYL-3,4-DIHYDRO-1H-QUINOLIN-2-ONE

Starting from 3-methyl-but-2-enoic acid {2-[2-(4-1,2-benzisothiazol-3-yl-piperazin-1-yl)-ethyl]-4-fluoro-phenyl}-amide (1.73 g, 3.95 mmol) and following the procedure as outlined in Example 1, 670 mg of 8-[2-(4-1,2-benzisothiazol-3-yl-piperazin-1-yl)-ethyl]-6-fluoro-4,4-dimethyl-3,4-dihydro-1H-quinolin-2-one hydrochloride was isolated as an off white powder in 100% purity © 254 nm; LC/MS (APCI): 439 [M+H]⁺; mp 298° C. ¹HNMR (400 MHz, DMSO-D₆) δ 8.08 (m, 2H), 7.55 (t, J=7.6 Hz, 1H), 7.43 (t, J=7.6 Hz, 1H), 7.06 (dd, J=9.5, 2.6 Hz, 1H), 6.96 (dd, J=9.5, 2.6 Hz, 1H), 4.08 (d, J=13.4 Hz, 2H), 3.65 (d, J=11.7 Hz, 2H), 3.44 (t, J=12.3 Hz, 2H), 3.30 (m, 4H), 3.08 (m, 2H), 2.31 (s, 2H), 1.18 (s, 6H).

### Preparation 12

### {2-[2-(4-1,2-Benzisothiazol-3-yl-piperazin-1-yl)-ethyl]-phenyl}-3-chloro-propionamide

2-[2-(4-1,2-Benzisothiazol-3-yl-piperazin-1-yl)-ethyl]-phenylamine (1.5 g, 4.43 mmol) was dissolved in 100 mL tetrahydrofuran (THF) and triethylamine was added (0.62 mL, 4.43 mmol). 3-Chloropropionyl chloride (0.45 mL, 4.66 mmol) was added under stirring and the reaction stirred at 0° C. for 45 min. The reaction mixture was concentrated under nitrogen and dissolved in 150 mL methylene chloride and then washed with water. The organic layer was concentrated and evaluated by LCMS. The mixture was purified by MPLC (medium pressure liquid chromatography) using a Biotage 40s prepacked silica gel cartridge eluting with 3% methanol in methylene chloride. {2-[2-(4-1,2-benzisothiazol-3-yl-piperazin-1-yl)-ethyl]-phenyl}-3-chloro-propionamide (0.83 g) was isolated in 100% purity @ 254 nm; LC/MS (APCI): 429 [M+H]⁺.

### Example 4

### 8-[2-(4-1,2-BENZISOTHIAZOL-3-YL-PIPERAZIN-1-YL)-ETHYL]-3,4-DIHYDRO-1H-QUINOLIN-2-ONE HYDROCHLORIDE

Starting from {2-[2-(4-1,2-benzisothiazol-3-yl-piperazin-1-yl)-ethyl]-phenyl}-3-chloro-propionamide (0.83 g, 1.94 mmol) and following the procedure as outlined in Example 1, 270 mg of 8-[2-(4-1,2-benzisothiazol-3-yl-piperazin-1-yl)-ethyl]-3,4-dihydro-1H-quinolin-2-one hydrochloride was isolated as a tan solid in 100% purity @ 254 nm; LCMS (APCI): 393 [M+H]⁺; mp 251° C. ¹HNMR (400 MHz, DMSO-D₆) δ 8.08 (m, 2H), 7.55 (t, 1H), 7.43 (t, 1H), 7.06 (m, 1H), 6.96 (m, 2H), 4.08 (d, J=13.4 Hz, 2H), 3.73 (m, 4H), 3.31 (m, 8H), 3.05 (m, 2H).

### Preparation 13

### {2-[2-(4-1,2-Benzisothiazol-3-yl-piperazin-1-yl)-ethyl]-phenyl}-3-chloro-2,2-dimethyl-propionamide

Starting from 2-[2-(4-1,2-benzisothiazol-3-yl-piperazin-1-yl)-ethyl]-phenylamine (338 mg, 1 mmol) and 3-chloropivaloyl chloride and following the procedure as outlined in Preparation 12, 442 mg of {2-[2-(4-1,2-benzisothiazol-3-yl-piperazin-1-yl)-ethyl]-phenyl}-3-chloro-2,2-dimethyl-propionamide was isolated as a white solid. MS (APCI): 457 [M+H]⁺.

### Example 5

### 8-[2-(4-1,2-BENZISOTHIAZOL-3-YL-PIPERAZIN-1-YL)-ETHYL]-3,3-DIMETHYL-3,4-DIHYDRO-1H-QUINOLIN-2-ONE

Starting from {2-[2-(4-1,2-benzisothiazol-3-yl-piperazin-1-yl)-ethyl]-phenyl}-3-chloro-2,2-dimethyl-propionamide (0.20 g, 0.44 mmol) and following the procedure as outlined in Example 1, 15 mg of 8-[2-(4-1,2-Benzisothiazol-3-yl-piperazin-1-yl)-ethyl]-3,3-dimethyl-3,4-dihydro-1H-quinolin-2-one was isolated as an off white solid in 100% purity @ 254 nm; LCMS (APCI): 421 [M+H]⁺; mp 95° C. ¹HNMR (400 MHz, DMSO-D₆) δ 8.08 (m, 2H), 7.51 (m, 1H), 7.38 (m, 1H), 6.97 (m, 2H), 6.83 (m, 1H), 3.49 (m, 4H), 2.76-2.44 (band, 10H), 0.97 (m, 6H).

### Preparation 14

### N-{2-[2-(4-Benzo[d]isothiazol-3-yl-piperazin-1-yl)-ethyl]-phenyl}-3-phenyl-acrylamide

Starting from 2-[2-(4-1,2-benzisothiazol-3-yl-piperazin-1-yl)-ethyl]-phenylamine (5 g, 14.8 mmol) and cinnamoyl chloride (2.58 g, 15.5 mmol) and following the procedure as outlined in Preparation 12, 6.8 g of N-{2-[2-(4-benzo[di]isothiazol-3-yl-piperazin-1-yl)-ethyl]-phenyl}-3-phenyl-acrylamide was isolated as a white powder. LCMS (APCI): 469 [M+H]⁺.

### Example 6

### 8-[2-(4-1,2-BENZISOTHIAZOL-3-YL-PIPERAZIN-1-YL)-ETHYL]-4-PHENYL-3,4-DIHYDRO-1H-QUINOLIN-2-ONE

Starting from N-{2-[2-(4-benzo[d]isothiazol-3-yl-piperazin-1-yl)-ethyl]-phenyl}-3-phenyl-acrylamide (6.7 g, 14.3 mmol) and following the procedure as outlined in Example 1, 1.53 g of 8-[2-(4-1,2-benzisothiazol-3-yl-piperazin-1-yl)-ethyl]-4-phenyl-3,4-dihydro-1H-quinolin-2-one was isolated as a white powder in 100% purity @ 254 nm; LC/MS (APCI): 469 [M+H]⁺; mp 227° C. ¹HNMR (400 MHz, DMSO-D₆) δ 8.12 (m, 2H), 7.64 (m, 3H), 7.42 (m, 2H), 7.2 6 (m, 2H), 7.1 5 (m, 2H), 6.92 (m, 1H), 4.10 (m, 2H), 3.65 (m, 2H), 3.45 (m, 8H), 3.13 (m, 2H).

### Example 7

### 8-[2-(4-BENZO[D]ISOTHIAZOL-3-YL-PIPERAZIN-1-YL)-ETHYL]-4,4-DIMETHYL-1,2,3,4-TETRAHYDRO-QUINOLINE

A dried, 3-necked round bottom flask, equipped with a thermometer under N₂ was charged with the 8-[2-(4-1,2-benzisothiazol-3-yl-piperazin-1-yl)-ethyl]-4,4-dimethyl-3,4-dihydro-1H-quinolin-2-one (3.41 g, 6.9 mmol) in toluene (250 mL). The flask was placed in an ice water bath and 2M borane methyl sulfide complex in toulene (5.52 mL, 11 mmol) was added slowly, maintaining the temperature below 20° C. The reaction stirred at reflux for two days and was monitored by Mass Spec (MS). The reaction was quenched at 0° C. slowly with 10% sodium carbonate (Na₂CO₃). This was heated to reflux until the complexes broke up-overnight. The mixture was concentrated and the residue was taken up in CH₂Cl₂ and washed with water. The organic layers were collected and the material was dried over sodium sulfate (Na₂SO₄), filtered, concentrated then chromatographed on an MPLC using a Biotage 40s prepacked silica gel cartridge eluting 50% CH₂Cl₂ in ethyl acetate to 100% ethyl acetate gradient over 1 h. 703 mg of 8-[2-(4-benzo[d]isothiazol-3-yl-piperazin-1-yl)-ethyl]-4,4-dimethyl-1,2,3,4-tetrahydro-quinoline was isolated as a powder in 91.7% purity @ 254 nm; LC/MS (APCI): 407.1 [M+H]⁺. ¹H NMR (400 MHz, CDCl₃) δ ppm 1.3 (s, 6H) 1.7 (m, 2H) 2.7 (s, 4H) 2.8 (m, 4H) 3.3 (m, 2H) 3.6 (m, 4H) 6.6 (t, J=7.4 Hz, 1H) 6.9 (dd, J=7.3, 1.5 Hz, 1H) 7.1 (dd, J=7.7, 1.6 Hz, 1H) 7.4 (ddd, J=8.1, 7.0, 1.0 Hz, 1H) 7.5 (td, J=7.6, 1.0 Hz, 1H) 7.8 (d, J=8.3 Hz, 1H) 7.9 (d, J=8.3 Hz, 1H).

### Example 8

### 1-{8-[2-(4-BENZO[D]ISOTHIAZOL-3-YL-PIPERAZIN-1-YL)-ETHYL]-4,4-DIMETHYL-3,4-DIHYDRO-2H-QUINOLIN-1-YL}-ETHANONE

8-[2-(4-Benzo[d]isothiazol-3-yl-piperazin-1-yl)-ethyl]-4,4-dimethyl-1,2,3,4-tetrahydro-quinoline (108 mg, 0.267 mmol) was dissolved in 4 mL tetrahydrofuran (THF) and triethylamine (55.8 μL, 0.4 mmol) was added. Acetyl chloride (20.8 μL, 0.29 mmol) was added under stirring and the reaction stirred overnight at room temperature. The reaction mixture was concentrated under nitrogen and dissolved in methylene chloride and then washed with water. The organic layer was concentrated and evaluated by LCMS. The mixture was purified by MPLC (medium pressure liquid chromatography) using a Biotage 40s prepacked silica gel cartridge eluting 50% CH₂Cl₂ in ethyl acetate to 100% ethyl acetate gradient over 1 h. 1-{8-[2-(4-Benzo[d]isothiazol-3-yl-piperazin-1-yl)-ethyl]-4,4-dimethyl-3,4-dihydro-2h-quinolin-1-yl}-ethanone (44 mg) was isolated in 100% purity @ 254 nm; LC/MS (APCI): 449 [M+H]⁺. ¹H NMR (400 MHz, CDCl₃) δ ppm 1.1 (s, 3H) 1.3 (d, J=6.8 Hz, 4H) 2.7 (m, 6H) 2.9 (s, 1H) 3.5 (s, 4H) 4.7 (m, 1H) 7.2 (m, 3H) 7.3 (m, 1H) 7.4 (ddd, J=8.1, 7.0, 1.0 Hz, 1H) 7.8 (d, J=8.3 Hz, 1H) 7.9 (d, J=8.1 Hz, 1H).

The amides of Examples 9-12 were synthesized in combinatorial library format following the steps outlined in Example 8 on a 0.267 mmol scale using 8-[2-(4-benzo[d]isothiazol-3-yl-piperazin-1-yl)-ethyl]-4,4-dimethyl-1,2,3,4-tetrahydro-quinoline and appropriate acid chloride starting materials. The crude products were purified by MPLC using a Biotage 40s prepacked silica gel cartridge eluting 50% CH₂Cl₂ in ethyl acetate to 100% ethyl acetate gradient over 1 hour (h).

### Example 9

### {8-[2-(4-BENZO[D]ISOTHIAZOL-3-YL-PIPERAZIN-1-YL)-ETHYL]-4,4-DIMETHYL-3,4-DIHYDRO-2H-QUINOLIN-1-YL}-PHENYL-METHANONE

Isolated in 93.51% purity @ 254 nm; LCMS (APCI): 511.1 [M+H]⁺.

### Example 10

### {8-[2-(4-BENZO[D]ISOTHIAZOL-3-YL-PIPERAZIN-1-YL)-ETHYL]-4,4-DIMETHYL-3,4-DIHYDRO-2H-QUINOLIN-1-YL}-(3-METHOXY-PHENYL)-METHANONE

Isolated in 100% purity @ 254 nm; LCMS (APCI): 555 [M+H]⁺.

### Example 11

### {8-[2-(4-BENZO[D]ISOTHIAZOL-3-YL-PIPERAZIN-1-YL)-ETHYL]-4,4-DIMETHYL-3,4-DIHYDRO-2H-QUINOLIN-1-YL}-(2,5-DIMETHOXY-PHENYL)-METHANONE

Isolated in 93.92% purity @ 254 nm; LCMS (APCI): 585 [M+H]⁺.

### Example 12

### {8-[2-(4-BENZO[D]ISOTHIAZOL-3-YL-PIPERAZIN-1-YL)-ETHYL]-4,4-DIMETHYL-3,4-DIHYDRO-2H-QUINOLIN-1-YL}-CYCLOHEXYL-METHANONE

Isolated in 86% purity @ 254 nm; LCMS (APCI): 517 [M+H]⁺.

### Example 13

### 8-[2-(4-BENZO[D]ISOTHIAZOL-3-YL-PIPERAZIN-1-YL)-ETHYL]-1-(1,2-DIMETHYL-1H-IMIDAZOLE-4-SULFONYL)-4,4-DIMETHYL-1,2,3,4-TETRAHYDRO-QUINOLINE

8-[2-(4-Benzo[d]isothiazol-3-yl-piperazin-1-yl)-ethyl]-4,4-dimethyl-1,2,3,4-tetrahydro-quinoline (108 mg, 0.267 mmol) was dissolved in 4 mL pyridine. 1,2-Dimethyl 1H imidazole-4-sulfonyl chloride (57.2 mg, 0.29 mmol) was added under stirring and the reaction stirred at overnight at 40° C. The reaction mixture was concentrated under nitrogen and dissolved in methylene chloride and then washed with water. The organic layer was concentrated and evaluated by LCMS. The mixture was purified by MPLC using a Biotage 40s prepacked silica gel cartridge eluting 50% CH₂Cl₂ in ethyl acetate to 100% ethyl acetate gradient over 1 h. 8-[2-(4-Benzo[d]isothiazol-3-yl-piperazin-1-yl)-ethyl]-1-(1,2-dimethyl-1H-imidazole-4-sulfonyl)-4,4-dimethyl-1,2,3,4-tetrahydro-quinoline (7 mg) was isolated in 100% purity @ 254 nm; LC/MS (APCI): 565 [M+H]⁺.

### Example 14

### 6-FLUORO-4,4-DIMETHYL-8-{2-[4-(1-OXO-1H-1 [symbol] ⁴-BENZO[D]ISOTHIAZOL-3-YL)-PIPERAZIN-1-YL]-ETHYL}-3,4-DIHYDRO-1H-QUINOLIN-2-ONE

The 8-[2-(4-1,2-benzisothiazol-3-yl-piperazin-1-yl)-ethyl]-6-fluoro-4,4-dimethyl-3,4-dihydro-1H-quinolin-2-one (240 mg, 0.55 mmol) was diluted in methylene chloride (100 mL) and 2-benzenesulfonyl-3-phenyl-oxaziridine (186 mg, 0.712 mmol) was added slowly with stirring. After 6 h the reaction was concentrated then chromatographed on an MPLC using a Biotage 40s prepacked silica gel cartridge eluting 100% CH₂Cl₂ to 10% methanol in CH₂Cl₂ over 1 h. 6-Fluoro-4,4-dimethyl-8-{2-[4-(1−oxo-1H-1λ⁴-benzo[d]isothiazol-3-yl)-piperazin-1-yl]-ethyl}-3,4-dihydro-1H-quinolin-2-one (30 mg) was isolated as a powder in 100% purity @ 254 nm; LC/MS (APCI): 455.2 [M+H]⁺; mp 235° C. ¹H NMR (400 MHz, CDCl₃) δ ppm 1.3 (s, 6H) 2.4 (s, 2H) 2.7 (s, 2H) 2.8 (s, 6H) 4.2 (s, 4H) 6.7 (d, J=8.5 Hz, 1H) 6.9 (d, J=7.6 Hz, 1H) 7.6 (d, J=7.8 Hz, 2H) 7.8 (d, J=7.6 Hz, 1H) 8.0 (d, J=6.8 Hz, 1H) 11.4 (s, 1H).

### Example 15

### 8-[2-(4-BENZO[D]ISOTHIAZOL-3-YL-PIPERAZIN-1-YL)-ETHYL]-6-FLUORO-4,4-DIMETHYL-1,2,3,4-TETRAHYDRO-QUINOLINE

Starting from 8-[2-(4-1,2-benzisothiazol-3-yl-piperazin-1-yl)-ethyl]-6-fluoro-4,4-dimethyl-3,4-dihydro-1H-quinolin-2-one (3.5 g, 7.99 mmol) and following the procedure as outlined in Example 7, 2.55 g of 8-[2-(4-benzo[d]isothiazol-3-yl-piperazin-1-yl)-ethyl]-6-fluoro-4,4-dimethyl-1,2,3,4-tetrahydro-quinoline was isolated as a white powder in 100% purity @ 254 nm; LC/MS (APCI): 424 [M+H]⁺; mp 264° C. ¹H NMR (400 MHz, CDCl₃) 8 ppm 1.2 (s, 6H) 1.7 (m, 2H) 3.1 (s, 6H) 3.4 (m, 4H) 4.1 (s, 4H) 6.5 (dd, J=8.5, 2.9 Hz, 1H) 6.8 (dd, J=10.3, 2.9 Hz, 1H) 7.4 (m, 1H) 7.5 (td, J=7.6, 1.1 Hz, 1H) 7.8 (m, 2H).

### Example 16

### 6-FLUORO-8-{2-[4-(5-FLUORO-BENZO[D]ISOTHIAZOL-3-YL)-PIPERAZIN-1-YL]-ETHYL}-4,4-DIMETHYL-3,4-DIHYDRO-1H-QUINOLIN-2-ONE

3-Methyl-but-2-enoic acid (4-fluoro-2-{2-[4-(5-fluorobenzo[d]isothiazol-3-yl)-piperazin-1-yl]-ethyl}-phenyl)-amide (2.17 g, 4.75 mmol) was dissolved in 100 mL of methylene chloride and methanesulfonic acid (0.924 mL, 14.26 mmol) was added slowly. The acetamide mixture was slowly added to a stirred suspension of the aluminum chloride (5.07 g, 38 mmol) in methylene chloride. The reaction stirred at room temperature overnight and was slowly poured into ice water then was made basic using 2 M sodium hydroxide (NaOH). The solution was extracted into methylene chloride and the organic layer was washed with water and dried over sodium sulfate (Na₂SO₄). The organic was filtered and concentrated. A small sample was triturated in ethyl acetate and recrystallized in acetonitrile to purify for submission; 65 mg of 6-fluoro-8-{2-[4-(5-fluoro-benzo[d]isothiazol-3-yl)-piperazin-1-yl]-ethyl}-4,4-dimethyl-3,4-dihydro-1H-quinolin-2-one was isolated as a powder in 98.5% purity @ 254 nm; LC/MS (APCI): 457 [M+H]⁺; mp 191° C. ¹H NMR (400 MHz, CDCl₃) δ ppm 1.3 (m, 6H) 2.4 (s, 2H) 2.7 (s, 2H) 2.8 (s, 2H) 2.8 (m, 4H) 3.7 (m, 4H) 6.7 (dd, J=8.9, 2.8 Hz, 1H) 6.9 (dd, J=9.4, 2.8 Hz, 1H) 7.23 (m, 1H) 7.5 (dd, J=9.3, 2.2 Hz, 1H) 7.7 (dd, J=8.8, 4.6 Hz, 1H) 11.3 (s, 1H).

### Preparation 15

### 6-Fluoro-8-{2-[4-(5-fluoro-benzordlisothiazol-3-yl)-Piperazin-1-yl]-ethyl}-4,4-dimethyl-1,2,3,4-tetrahydro-quinoline

Starting from 6-fluoro-8-{2-[4-(5-fluoro-benzo[d]isothiazol-3-yl)-piperazin-1-yl]-ethyl}-4,4-dimethyl-3,4-dihydro-1H-quinolin-2-one (1.76 g, 3.85 mmol) and following the procedure as outlined in Example 7, 800 mg of 6-fluoro-8-{2-[4-(5-fluoro-benzo[d]isothiazol-3-yl)-piperazin-1-yl]-ethyl}-4,4-dimethyl-1,2,3,4-tetrahydro-quinoline was isolated. MS (APCI): 443.2 [M+H]⁺.

### Example 17

### 1-(6-FLUORO-8-{2-[4-(5-FLUORO-BENZO[D]ISOTHIAZOL-3-YL)-PIPERAZIN-1-YL]-ETHYL}-4,4-DIMETHYL-3,4-DIHYDRO-2H-QUINOLIN-1-YL)-ETHANONE

Starting from 6-fluoro-8-{2-[4-(5-fluoro-benzo[d]isothiazol-3-yl)-piperazin-1-yl]-ethyl}-4,4-dimethyl-1,2,3,4-tetrahydro-quinoline (800 mg, 1.81 mmol) and acetyl choride (0.135 mL, 1.89 mmol) following the procedure as outlined in Example 8, 610 mg of 1-(6-fluoro-8-{2-[4-(5-fluoro-benzo[d]isothiazol-3-yl)-piperazin-1-yl]-ethyl}-4,4-dimethyl-3,4-dihydro-2H-quinolin-1-yl)-ethanone was isolated as an HCl salt in 100% purity @ 254 nm; LC/MS (APCI): 485.1 [M+H]⁺; mp 128° C. ¹H NMR (400 MHz, CDCl₃) δ ppm 1.2 (m, 3H) 1.3 (s, 3H) 2.4 (d, J=14.4 Hz, 7H) 3.0 (s, 1H) 3.2 (s, 4H) 3.5 (s, 1H) 3.6 (s, 2H) 4.1 (s, 4H) 6.8 (s, 1H) 7.0 (dd, J=9.3, 1.7 Hz, 1H) 7.3 (t, J=8.4 Hz, 1H) 7.5 (d, J=7.8 Hz, 1H) 7.8 (dd, J=8.9, 4.5 Hz, 1H).

### Example 18

### 8-[3-(4-BENZO[D]ISOTHIAZOL-3-YL-PIPERAZIN-1-YL)-PROPYL]-4,4-DIMETHYL-3,4-DIHYDRO-1H-QUINOLIN-2-ONE

Starting from 3-methyl-but-2-enoic acid {2-[3-(4-benzo[d]isothiazol-3-yl-piperazin-1-yl)-propyl]-phenyl}-amide (550 mg, 1.27 mmol) and following the procedure as outlined in Example 1, 174 mg of 8-[3-(4-benzo[d]isothiazol-3-yl-piperazin-1-yl)-propyl]-4,4-dimethyl-3,4-dihydro-1H-quinolin-2-one was isolated as pink crystals in 100% purity @ 254 nm; LC/MS (APCI): 435 [M+H]⁺; mp 166° C. ¹H NMR (400 MHz, CDCl₃) δ ppm 1.6 (s, 6H) 1.9 (s, 2H) 2.3 (s, 2H) 2.4 (s, 2H) 2.7 (d, J=6.8 Hz, 4H) 2.7 (s, 2H) 3.9 (s, 4H) 7.0 (m, 2H) 7.2 (dd, J=7.4, 1.3 Hz, 1H) 7.3 (d, J=7.6 Hz, 1H) 7.4 (t, J=7.7 Hz, 1H) 7.8 (d, J=8.1 Hz, 1H) 7.9 (d, 1H).

Preparation 16

### 8-[3-(4-Benzo[d]isothiazol-3-yl-piperazin-1-yl)-propyl]-4,4-dimethyl-1,2,3,4-tetrahydro-quinoline

Starting from 8-[3-(4-benzo[d]isothiazol-3-yl-piperazin-1-yl)-propyl]-4,4-dimethyl-3,4-dihydro-1H-quinolin-2-one (2.1 g, 4.84 mmol) and following the procedure as outlined in Example 7, 810 mg of 8-[3-(4-benzo[d]isothiazol-3-yl-piperazin-1-yl)-propyl]-4,4-dimethyl-1,2,3,4-tetrahydro-quinoline was isolated. MS (APCI): 421.2 [M+H]⁺.

### Example 19

### 1-{8-[3-(4-BENZO[D]ISOTHIAZOL-3-YL-PIPERAZIN-1-YL)-PROPYL]-4,4-DIMETHYL-3,4-DIHYDRO-2H-QUINOLIN-1-YL}-ETHANONE

Starting from 8-[3-(4-benzo[d]isothiazol-3-yl-piperazin-1-yl)-propyl]-4,4-dimethyl-1,2,3,4-tetrahydro-quinoline (810 mg, 1.93 mmol) and acetyl choride (0.206 mL, 2.89 mmol) following the procedure as outlined in Example 8, 62 mg of 1-{8-[3-(4-benzo[d]isothiazol-3-yl-piperazin-1-yl)-propyl]-4,4-dimethyl-3,4-dihydro-2H-quinolin-1-yl}-ethanone was isolated as an HCl salt in 100% purity @ 254 nm; LC/MS (APCI): 463.1 [M+H]⁺; mp 109° C. ¹H NMR (400 MHz, CDCl₃) δ ppm 1.2 (d, 3H) 1.3 (d, J=7.8 Hz, 3H) 1.9 (s, 2H) 2.2 (s, 2H) 2.3 (s, 2H) 2.4 (s, 4H) 2.7 (s, 2H) 3.0 (s, 3H) 3.5 (s, 2H) 4.1 (s, 4H) 7.2 (d, J=8.1 Hz, 3H) 7.4 (s, 1H) 7.5 (d, J=7.8 Hz, 1H) 7.8 (d, J=7.8 Hz, 2H).

### Example 20

### 8-[2-(4-BENZO[D]ISOTHIAZOL-3-YL-PIPERAZIN-1-YL)-ETHYL]-4,4,5-TRIMETHYL-3,4-DIHYDRO-1H-QUINOLIN-2-ONE

Starting from 3-methyl-but-2-enoic acid {2-[2-(4-benzo[d]isothiazol-3-yl-piperazin-1-yl)-ethyl]-5-methyl-phenyl}-amide (1.69 g, 3.89 mmol) and following the procedure as outlined in Example 16, 348 mg of 8-[2-(4-benzo[d]isothiazol-3-yl-piperazin-1-yl)-ethyl]-4,4,5-trimethyl-3,4-dihydro-1H-quinolin-2-one was isolated as a white foam in 100% purity @ 254 nm; LC/MS (APCI): 435.1 [M+H]⁺; mp 129° C. ¹H NMR (400 MHz, CDCl₃) 8 ppm 1.4 (s, 6H) 2.4 (s, 2H) 2.5 (s, 3H) 2.7 (s, 2H) 2.8 (d, J=4.9 Hz, 5H) 2.8 (s, 1H) 3.8 (m, 4H) 6.7 (d, J=7.6 Hz, 1H) 6.9 (d, J=7.8 Hz, 1H) 7.3 (t, J=7.7 Hz, 1H) 7.4 (t, J=7.4 Hz, 1H) 7.8 (d, J=8.3 Hz, 1H) 7.9 (d, J=8.5 Hz, 1H) 11.4 (s, 1H).

### Preparation 17

### 8-[3-(4-Benzo[d]isothiazol-3-yl-piperazin-1-yl)-Propyl]-4,4-dimethyl-1,2,3,4-tetrahydro-quinoline

Starting from 8-[2-(4-benzo[d]isothiazol-3-yl-piperazin-1-yl)-ethyl]-4,4,5-trimethyl-3,4-dihydro-1H-quinolin-2-one (2 g, 4.6 mmol) and following the procedure as outlined in Example 7, 1.57 g of 8-[2-(4-benzo[d]isothiazol-3-yl-piperazin-1-yl)-ethyl]-4,4,5-trimethyl-1,2,3,4-tetrahydro-quinoline was isolated. MS (APCI): 421.1 [M+H]⁺.

### Example 21

### 1-{8-[2-(4-BENZO[D]ISOTHIAZOL-3-YL-PIPERAZIN-1-YL)-ETHYL]-4,4,5-TRIMETHYL-3,4-DIHYDRO-2H-QUINOLIN-1-YL}-ETHANONE

Starting from 8-[2-(4-benzo[d]isothiazol-3-yl-piperazin-1-yl)-ethyl]-4,4,5-trimethyl-1,2,3,4-tetrahydro-quinoline (1.3 g, 3.1 mmol) and acetyl choride (0.330 mL, 4.6 mmol) following the procedure as outlined in Example 8, 622 mg of 1-{8-[2-(4-benzo[d]isothiazol-3-yl-piperazin-1-yl)-ethyl]-4,4,5-trimethyl-3,4-dihydro-2H-quinolin-1-yl}-ethanone was isolated as an HCl salt in 100% purity @ 254 nm; LC/MS (APCI): 463.1 [M+H]⁺; mp 132° C. ¹H NMR (400 MHz, CDCl₃) δ ppm 1.2 (s, 1H) 1.4 (m, 5H) 1.8 (m, 3H) 2.3 (s, 2H) 2.5 (s, 4H) 2.9 (s, 2H) 3.2 (s, 4H) 3.5 (s, 3H) 4.1 (s, 4H) 7.0 (s, 2H) 7.4 (s, 1H) 7.5 (s, 1H) 7.8 (d, J=7.1 Hz, 2H).

### Preparation 18

### 8-[2-(4-Benzo[d]isothiazol-3-yl-piperazin-1-yl)-ethyl]-4-Phenyl-1,2,3,4-tetrahydro-quinoline

Starting from 8-[2-(4-1,2-benzisothiazol-3-yl-piperazin-1-yl)-ethyl]-4-phenyl-3,4-dihydro-1H-quinolin-2-one (970 mg, 2.08 mmol) and following the procedure as outlined in Example 7, 140 mg of 8-[2-(4-benzo[d]isothiazol-3-yl-piperazin-1-yl)-ethyl]-4-phenyl-1,2,3,4-tetrahydro-quinoline was isolated. MS (APCI): 457.2 [M+H]⁺.

### Example 22

### 1-{8-[2-(4-BENZO[D]ISOTHIAZOL-3-YL-PIPERAZIN-1-YL)-ETHYL]-4-PHENYL-3,4-DIHYDRO-2H-QUINOLIN-1-YL}-ETHANONE

Starting from 8-[2-(4-benzo[d]isothiazol-3-yl-piperazin-1-yl)-ethyl]-4-phenyl-1,2,3,4-tetrahydro-quinoline (160 mg, 0.35 mmol) and acetyl choride (0.0375 mL, 0.53 mmol) following the procedure as outlined in Example 8, 75 mg of 1-{8-[2-(4-benzo[d]isothiazol-3-yl-piperazin-1-yl)-ethyl]-4-phenyl-3,4-dihydro-2H-quinolin-1-yl}-ethanone was isolated as an HCl salt in 94.5% purity @ 254 nm; LC/MS (APCI): 499.1 [M+H]⁺; mp 97° C. ¹H NMR (400 MHz, CDCl₃) δ ppm 1.8 (s, 3H) 1.8 (s, 2H) 2.0 (s, 1H) 2.4 (s, 2H) 2.6 (s, 3H) 2.8 (s, 4H) 2.9 (s, 2H) 3.7 (s, 1H) 4.1 (s, 2H) 7.1 (s, 5H) 7.2 (s, 1H) 7.3 (s, 2H) 7.5 (s, 2H) 7.8 (s, 2H).

### Example 23

### 1-{8-[2-(4-BENZO[D]ISOTHIAZOL-3-YL-PIPERAZIN-1-YL)-ETHYL]-6-FLUORO-4,4-DIMETHYL-3,4-DIHYDRO-2H-QUINOLIN-1-YL}-ETHANONE

Starting from 8-[2-(4-benzo[d]isothiazol-3-yl-piperazin-1-yl)-ethyl]-6-fluoro-4,4-dimethyl-1,2,3,4-tetrahydro-quinoline (250 mg, 0.59 mmol) and acetyl choride (0.050 mL, 0.71 mmol) following the procedure as outlined in Example 8, 82 mg of 1-{8-[2-(4-benzo[d]isothiazol-3-yl-piperazin-1-yl)-ethyl]-6-fluoro-4,4-dimethyl-3,4-dihydro-2H-quinolin-1-yl}-ethanone was isolated as a white foam in 100% purity @ 254 nm; LC/MS (APCI): 467.1 [M+H]⁺. ¹H NMR (400 MHz, CDCl₃) δ ppm 1.1 (s, 2H) 1.2 (m, 3H) 1.3 (d, J=7.1 Hz, 3H) 1.9 (m, 3H) 2.1 (s, 2H) 2.7 (m, 6H) 2.9 (s, 1H) 3.0 (s, 1H) 3.5 (m, 4H) 6.9 (m, 2H) 7.3 (td, J=7.6, 1.0 Hz, 1H) 7.5 (td, J=7.5, 1.1 Hz, 1H) 7.8 (d, J=8.1 Hz, 1H) 7.9 (m, 1H).

### Example 24

### 8-[2-(4-BENZO[D]ISOXAZOL-3-YL-PIPERAZIN-1-YL)-ETHYL]-4,4,5-TRIMETHYL-3,4-DIHYDRO-1H-QUINOLIN-2-ONE

Starting from 3-methyl-but-2-enoic acid {2-[2-(4-benzo[d]isoxazol-3-yl-piperazin-1-yl)-ethyl]-5-methyl-phenyl}-amide (1.82 g, 4.35 mmol) and following the procedure as outlined in Example 16, 1.6 g of 8-[2-(4-benzo[d]isoxazol-3-yl-piperazin-1-yl)-ethyl]-4,4,5-trimethyl-3,4-dihydro-1H-quinolin-2-one was isolated as a white solid in 100% purity @ 254 nm; LC/MS (APCI): 419.2 [M+H]⁺; mp 170° C. ¹H NMR (400 MHz, CDCl₃) δ ppm 1.4 (s, 6H) 2.4 (s, 2H) 2.5 (d, J=2.7 Hz, 3H) 2.7 (s, 2H) 2.8 (s, 6H) 3.8 (m, 4H) 6.7 (d, J=8.3 Hz, 1H) 6.9 (d, J=7.6 Hz, 1H) 7.2 (s, 1H) 7.4 (m, 2H) 7.7 (d, J=8.1 Hz, 1H).

### Example 25

### 8-[2-(4-BENZO[D]ISOXAZOL-3-YL-PIPERAZIN-1-YL)-ETHYL]-4,4,5-TRIMETHYL-1,2,3,4-TETRAHYDRO-QUINOLINE

Starting from 8-[2-(4-benzo[d]isoxazol-3-yl-piperazin-1-yl)-ethyl]-4,4,5-trimethyl-3,4-dihydro-1H-quinolin-2-one (1.42 g, 3.39 mmol) and following the procedure as outlined in Example 7, 1.33 g of 8-[2-(4-benzo[d]isoxazol-3-yl-piperazin-1-yl)-ethyl]-4,4,5-trimethyl-1,2,3,4-tetrahydro-quinoline was isolated as a white powder in 100% purity @ 254 nm; LC/MS (APCI): 405.2 [M+H]⁺; mp 135° C. ¹H NMR (400 MHz, CDCl₃) 8 ppm 1.4 (m, 6H) 2.4 (d, J=3.9 Hz, 3H) 2.7 (s, 4H) 2.7 (s, 6H) 3.2 (d, J=5.6 Hz, 2H) 3.6 (s, 4H) 6.4 (d, J=7.6 Hz, 1H) 6.7 (d, J=7.6 Hz, 1H) 7.2 (ddd, J=8.0, 6.4,1.5 Hz, 1H) 7.5 (m, 2H) 7.7 (d, J=8.1 Hz, 1H)

### Example 26

### 1-{8-[2-(4-BENZO[D]ISOXAZOL-3-YL-PIPERAZIN-1-YL)-ETHYL]-4,4,5-TRIMETHYL-3,4-DIHYDRO-2H-QUINOLIN-1-YL}-ETHANONE

Starting from 8-[2-(4-benzo[d]isoxazol-3-yl-piperazin-1-yl)-ethyl]-4,4,5-trimethyl-1,2,3,4-tetrahydro-quinoline (1.23 g, 3.04 mmol) and acetyl choride (0.325 mL, 4.57 mmol) following the procedure as outlined in Example 8, 452 mg of 1-{8-[2-(4-benzo[d]isoxazol-3-yl-piperazin-1-yl)-ethyl]-4,4,5-trimethyl-3,4-dihydro-2H-quinolin-1-yl}-ethanone was isolated as an HCl salt (fine white powder) in 100% purity @ 254 nm; LC/MS (APCI): 446.8 [M+H]⁺; mp 230° C. ¹H NMR (400 MHz, CDCl₃) δ ppm 1.4 (m, 6H) 1.8 (s, 2H) 1.8 (m, 2H) 2.3 (s, 2H) 2.5 (m, 3H) 2.9 (s, 2H) 3.1 (s, 4H) 3.4 (d, J=5.1 Hz, 1H) 3.5 (s, 1H) 3.6 (s, 1H) 4.1 (s, 4H) 7.0 (m, 2H) 7.3 (d, J=6.8 Hz, 1H) 7.5 (m, 2H) 7.6 (d, J=8.1 Hz, 1H).

### Example 27

### 8-[2-(4-BENZO[D]ISOXAZOL-3-YL-PIPERAZIN-1-YL)-ETHYL]-6-FLUORO-4,4-DIMETHYL-3,4-DIHYDRO-1H-QUINOLIN-2-ONE

Starting from 3-methyl-but-2-enoic acid {2-[2-(4-benzo[d]isoxazol-3-yl-piperazin-1-yl)-ethyl]-4-fluoro-phenyl}-amide (2.25 g, 5.33 mmol) and following the procedure as outlined in Example 16, 1.15 g of 8-[2-(4-benzo[d]isoxazol-3-yl-piperazin-1-yl)-ethyl]-6-fluoro-4,4-dimethyl-3,4-dihydro-1H-quinolin-2-one was isolated as an off white solid in 100% purity @ 254 nm; LC/MS (APCI): 422.8 [M+H]⁺; mp 230° C. ¹H NMR (400 MHz, DMSO-D₆) δ ppm 1.2 (s, 6H) 2.3 (s, 2H) 2.6 (s, 2H) 2.7 (s, 4H) 2.8 (s, 2H) 3.5 (s, 4H) 6.9 (s, 2H) 7.3 (s, 1H) 7.6 (s, 2H) 8.0 (s, 1H).

### Preparation 19

### 8-[2-(4-Benzordlisoxazol-3-yl-piperazin-1-yl)-ethyl]-6-fluoro-4,4-dimethyl-1,2,3,4-tetrahydro-quinoline

Starting from 8-[2-(4-benzo[d]isothiazol-3-yl-piperazin-1-yl)-ethyl]-4,4,5-trimethyl-3,4-dihydro-1H-quinolin-2- one (920 mg, 2.18 mmol) and following the procedure as outlined in Example 7, 900 mg of 8-[2-(4-benzo[d]isoxazol-3-yl-piperazin-1-yl)-ethyl]-6-fluoro-4,4-dimethyl-1,2,3,4-tetrahydro-quinoline was isolated. MS (APCI): 409.2 [M+H]⁺.

### Example 28

### 1-{8-[2-(4-BENZO[D]ISOXAZOL-3-YL-PIPERAZIN-1-YL)-ETHYL]-6-FLUORO-4,4-DIMETHYL-3.4-DIHYDRO-2H-QUINOLIN-1-YL}-ETHANONE

Starting from 8-[2-(4-benzo[d]isoxazol-3-yl-piperazin-1-yl)-ethyl]-6-fluoro-4,4-dimethyl-1,2,3,4-tetrahydro-quinoline (800 mg, 1.96 mmol) and acetyl choride (0.209 mL, 2.94 mmol) following the procedure as outlined in Example 8, 256 mg of 1-{8-[2-(4-benzo[d]isoxazol-3-yl-piperazin-1-yl)-ethyl]-6-fluoro-4,4-dimethyl-3,4-dihydro-2H-quinolin-1-yl}-ethanone was isolated as an HCl salt in 100% purity @ 254 nm; LC/MS (APCI): 451.1 [M+H]⁺; mp 112° C. ¹H NMR (400 MHz, CDCl₃) δ ppm 1.2 (s, 3H) 1.3 (s, 3H) 1.8 (d, J=12.7 Hz, 2H) 2.0 (d, J=5.4 Hz, 3H) 2.3 (s, 2H) 3.1 (s, 2H) 3.4 (s, 2H) 3.6 (s, 2H) 3.7 (m, 2H) 4.1 (s, 4H) 6.8 (s, 1H) 6.9 (d, J=9.3 Hz, 1H) 7.3 (s, 1H) 7.5 (m, 2H) 7.6 (s, 1H).

### Example 29

### 8-[2-(4-BENZO[D]ISOTHIAZOL-3-YL-PIPERAZIN-1-YL)-ETHYL]-4-METHYL-1H-QUINOLIN-2-ONE

In an open tube (8 mL) equipped with a stir bar, the ortho aniline (338 mg, 1.0 mmol), o-xylene (1 mL) and ethyl acetoacetate (140 μl, 1.1 mmol) were combined. The mixture was then warmed to 130° C. in an aluminum heating block for 2.5 h. (TLC and MS showed only a trace of remaining aniline.) Reaction was cooled and concentrated to dryness (light yellow oil). The crude amide was then treated with 1 mL of sulfuric acid and reaction was sealed and warmed to 80° C. for 1 h. The reaction was cooled and poured into water/ice. The pH was brought to neutral (˜7) with 50% NaOH. The ppt was filtered and dried to constant weight. The crude was then dissolved in 400:8:1 methylene chloride:ethanol:ammonium hydroxide (CH₂Cl₂:EtOH:NH₄OH) and loaded onto a silica gel cartridge and purified via MPLC, (silica cartridge, 40 g) eluting with gradient of methylene chloride to (100:8:1) CH₂Cl₂:EtOH:NH₄OH over a 1 h period, yielding pure product (99 mg, 24.5% yield). MS (APCI): 405 [M+H]⁺; ¹HNMR (400 MHz, DMSO-D₆) δ ppm 2.39 (s, 3H) 2.66 (m, 2H) 2.78 (m, 4H) 3.02 (m, 2H) 3.61 (m, 4H) 6.37 (s, 1H) 7.10 (m, 1H) 7.39 (m, 2H) 7.55 (m, 2H) 8.04 (d, J=8.30 Hz, 2H).

### Example 30

### 8-[2-(4-BENZO[D]ISOTHIAZOL-3-YL-PIPERAZIN-1-YL)-ETHYL]-3,4-DIMETHYL-1H-QUINOLIN-2-ONE

8-[2-(4-Benzo[d]isothiazol-3-yl-piperazin-1-yl)-ethyl]-3,4-dimethyl-1H-quinolin-2-one was prepared in a similar manner as example 29 using ethyl-2-methyl acetoacetate. (168 mg, 40.2% yield). MS (APCI): 419 [M+H]⁺; ¹H NMR (400 MHz, DMSO-D₆) δ ppm 2.06 (s, 3H) 2.36 (s, 3H) 2.45 (dt, J=3.66, 1.83 Hz, 15H) 2.65 (d, J=5.37 Hz, 2H) 2.76 (s, 4H) 3.00 (s, 2H) 3.28 (s, 5H) 3.61 (d, J=5.12 Hz, 4H) 7.05 (m, 1H) 7.27 (d, J=6.59 Hz, 1H) 7.39 (m, 1H) 7.52 (m, 1H) 7.58 (d, J=8.05 Hz, 1H) 8.02 (d, J=9.03 Hz, 2H)

### Example 31

### 8-[2-(4-BENZO[D]ISOTHIAZOL-3-YL-PIPERAZIN-1-YL)-ETHYL]-4-ETHYL-1H-QUINOLIN-2-ONE

8-[2-(4-Benzo[d]isothiazol-3-yl-piperazin-1-yl)-ethyl]-4-ethyl-1H-quinolin-2-one was prepared in a similar manner as example 29 using ethylpropionylacetate. (194 mg, 46.3% yield). MS (APCI): 419 [M+H]⁺; ¹H NMR (400 MHz, DMSO-D₆) δ ppm 1.21 (t, J=7.33 Hz, 3H) 2.47 (dt, J=3.66, 1.83 Hz, 17H) 2.66 (m, 2H) 2.79 (d, J=6.35 Hz, 5H) 2.81 (s, 1H) 3.02 (s, 2H) 3.61 (d, J=4.64 Hz, 4H) 6.33 (s, 1H) 7.09 (m, 1H) 7.35 (m, 1H) 7.41 (m, 1H) 7.54 (m, 1H) 7.62 (d, J=7.08 Hz, 1H) 8.04 (d, J=8.30 Hz, 2H).

### Example 32

### 6-[2-(4-BENZO[D]ISOTHIAZOL-3-YL-PIPERAZIN-1-YL)-ETHYL]-1,2,3,5-TETRAHYDRO-CYCLOPENTA[C]QUINOLIN-4-ONE

6-[2-(4-Benzo[d]isothiazol-3-yl-piperazin-1-yl)-ethyl]-1,2,3,5-tetrahydro-cyclopenta[c]quinolin-4-one was prepared in a similar manner as example 29 using 2-cyclopentanecarboxylic ethylester. (122 mg, 28.3% yield). MS (APCI): 431 [M+H]⁺; ¹H NMR (400 MHz, DMSO-D₆) δ ppm 2.06 (m, 2H) 2.47 (ddd, J=3.79, 1.83, 1.71 Hz, 11H) 2.66 (m, 2H) 2.75 (m, 6H) 3.04 (m, 4H) 3.62 (m, 4H) 7.08 (t, J=7.57 Hz, 1H) 7.32 (m, 1H) 7.40 (m, 2H) 7.54 (m, 1H) 8.04 (d, J=9.04 Hz, 2H).

### Example 33

### 8-[2-(4-BENZO[D]ISOTHIAZOL-3-YL-PIPERAZIN-1-YL)-ETHYL]-3-ETHYL-4-METHYL-1H-QUINOLIN-2-ONE

8-[2-(4-Benzo[d]isothiazol-3-yl-piperazin-1-yl)-ethyl]-3-ethyl-4-methyl-1H-quinolin-2-one was prepared in a similar manner as example 29 using ethyl-2-ethyl acetoacetate. (99 mg, 22.9% yield). MS (APCI): 433 [M+H]⁺; ¹H NMR (400 MHz, DMSO-D₆) δ ppm 0.99 (t, J=7.45 Hz, 3H) 2.64 (m, 4H) 2.79 (m, 4H) 3.02 (m, 2H) 3.63 (m, 4H) 7.07 (m, 1H) 7.29 (d, J=6.11 Hz, 1H) 7.41 (t, J=7.57 Hz, 1H) 7.54 (t, J=8.06 Hz, 1H) 7.60 (d, J=6.84 Hz, 1H) 8.04 (d, J=8.30 Hz, 2H).

### Example 34

### 8-[2-(4-BENZO[D]ISOTHIAZOL-3-YL-PIPERAZIN-1-YL)-ETHYL]-4-PROPYL-1H-QUINOLIN-2-ONE

8-[2-(4-Benzo[d]isothiazol-3-yl-piperazin-1-yl)-ethyl]-4-propyl-1H-quinolin-2-one was prepared in a similar manner as example 29 using ethyl butyrylacetate. (160 mg, 37.0% yield). MS (APCI): 433 [M+H]⁺; ¹H NMR (400 MHz, DMSO-D₆) δ ppm 0.93 (t, J=7.32 Hz, 3H) 1.60 (qd, J=7.48, 7.32 Hz, 2H) 2.64 (m, 2H) 2.74 (m, 6H) 3.00 (m, 2H) 3.60 (d, J=3.90 Hz, 4H) 6.30 (s, 1H) 7.07 (m, 1H) 7.33 (d, J=6.34 Hz, 1H) 7.41 (s, 1H) 7.51 (t, J=7.08 Hz, 1H) 7.60 (d, J=7.56 Hz, 1H) 8.02 (d, J=8.05 Hz, 2H).

### Example 35

### 8-[2-(4-BENZO[D]ISOTHIAZOL-3-YL-PIPERAZIN-1-YL)-ETHYL]-4-ISOPROPYL-1H-QUINOLIN-2-ONE

8-[2-(4-Benzo[d]isothiazol-3-yl-piperazin-1-yl)-ethyl]-4-isopropyl-1H-quinolin-2-one was prepared in a similar manner as example 29 using ethyl isobutyrylacetate. (160 mg, 37.0% yield). MS (APCI): 433 [M+H]⁺; ¹H NMR (400 MHz, DMSO-D₆) δ ppm 1.20 (d, J=6.83 Hz, 6H) 2.65 (m, 2H) 2.76 (m, 4H) 3.00 (m, 2H) 3.38 (m, 1H) 3.60 (d, J=4.88 Hz, 4H) 6.31 (s, 1H) 7.08 (m, 1H) 7.33 (d, J=6.34 Hz, 1H) 7.39 (t, J=7.69 Hz, 1H) 7.52 (m, 1H) 7.68 (d, J=8.30 Hz, 1H) 8.02 (d, J=9.03 Hz, 2H).

### Example 36

### 4-[2-(4-BENZO[D]ISOTHIAZOL-3-YL-PIPERAZIN-1-YL)-ETHYL]-7,8,9,10-TETRAHYDRO-5H-PHENANTHRIDIN-6-ONE

4-[2-(4-Benzo[d]isothiazol-3-yl-piperazin-1-yl)-ethyl]-7,8,9,10-tetrahydro-5H-phenanthridin-6-one was prepared in a similar manner as example 29 using ethyl cyclohexanone caboxylate. (56 mg, 12.6% yield). MS (APCI): 445 [M+H]⁺; ¹H NMR (400 MHz, DMSO-D₆) δ ppm 1.67 (d, J=8.79 Hz, 2H) 1.75 (d, J=4.15 Hz, 2H) 2.43 (m, 2H) 2.66 (m, 2H) 2.79 (d, J=4.88 Hz, 6H) 3.02 (m, 2H) 3.62 (d, J=4.88 Hz, 0.4H) 7.07 (m, 1H) 7.29 (d, J=6.84 Hz, 1H) 7.41 (t, J=8.06 Hz, 1H) 7.53 (t, J=8.06 Hz, 2H) 8.04 (d, J=8.79 Hz, 2H).

### Example 37

### 8-[2-(4-BENZO[D]ISOTHIAZOL-3-YL-PIPERAZIN-1-YL)-ETHYL]-4-TRIFLUOROMETHYL-1H-QUINOLIN-2-ONE

8-[2-(4-Benzo[d]isothiazol-3-yl-piperazin-1-yl)-ethyl]-4-trifluoromethyl-1H-quinolin-2-one was prepared in a similar manner as example 29 using ethyl-4,4,4-trifluoroacetate. (18 mg, 3.9% yield). MS (APCI): 459 [M+H]⁺; ¹H NMR (400 MHz, DMSO-D₆) δ ppm 2.71 (m, 2H) 2.82 (m, 4H) 3.08 (m, 2H) 3.65 (m, 4H) 6.95 (s, 1H) 7.22 (m, 1H) 7.41 (t, J=7.45 Hz, 1H) 7.54 (m, 3H) 8.05 (m, 2H).

### Example 38

### 8-[2-(4-BENZO[D]ISOTHIAZOL-3-YL-PIPERAZIN-1-YL)-ETHYL]-4-PHENYL-1H-QUINOLIN-2-ONE

8-[2-(4-Benzo[d]isothiazol-3-yl-piperazin-1-yl)-ethyl]-4-phenyl-1H-quinolin-2-one was prepared in a similar manner as example 29 using ethyl -4,4,4-trifluoroacetate. (33 mg, 7.0% yield). MS (APCI): 467 [M+H]⁺; ¹H NMR (400 MHz, DMSO-D₆) δ ppm 2.71 (m, 2H) 2.82 (m, 4H) 3.08 (m, 2H) 3.65 (d, J=5.13 Hz, 4H) 6.34 (s, 1H) 7.04 (m, 1H) 7.21 (d, J=8.06 Hz, 1H) 7.41 (dd, J=12.94, 7.08 Hz, 4H) 7.53 (m, 4H) 8.05 (m, 2H).

1. A compound of the formula 1 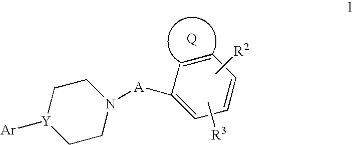
wherein Ar is 1,2-benzisothiazoyl, 1,2-benzisothiazoyl-1-oxide, 1,2-benzisothiazoyl-1-dioxide, 1,2-benzisoxazoyl, naphthyl, pyridyl, quinolyl, isoquinolyl, benzothiadiazolyl, benzotriazolyl, benzoxazolyl, benzoxazolonyl, phthalazinyl, indolyl, indanyl, 1H-indazoyl, or 3-indazolyl, and wherein Ar can optionally be substituted by one or more substituents, preferably from zero to four substituents, independently selected from halo, preferably chloro or fluoro, cyano, nitro, (C₁-C₆) alkyl optionally substituted with from one to three fluorine atoms and (C₁-C₆) alkoxy optionally substituted with from one to three fluorine atoms; with the proviso that Ar can not be attached to the piperazine ring via a phenyl ring of Ar;
Y is N or CH;
A is —(CH₂)ₙCH₂—, wherein n is an integer from one to four, wherein one of the CH₂ groups that is not adjacent to the piperazine nitrogen can optionally be replaced by an oxygen atom;
R² and R³ are independently selected from hydrogen, (C₁-C₆) alkyl optionally substituted with from one to three fluorine atoms, (C₁-C₆) alkoxy optionally substituted with from one to three fluorine atoms, halogen, nitro, cyano, amino, (C₁-C₆) alkylamino and di-(C₁-C₆) alkylamino; and
ring Q can be a saturated, unsaturated or aromatic five to seven membered monocyclic heterocyclic ring containing from one to three heteoratoms independently selected from oxygen, nitrogen and sulfur, and wherein ring Q can be optionally substituted with from one to four substituents, preferably with two or three substituents, independently selected from amino, oxo, hydroxy, (C₁-C₆) alkyl optionally substituted with from one to three fluorine atoms, (C₁-C₆) alkoxy optionally substituted with from one to three fluorine atoms, aryl, aryl-(C₁-C₆) alkyl-, (C₁-C₆) alkenyl optionally substituted with from one to three fluorine atoms, heteroaryl and heteroaryl-(C₁-C₆) alkyl-, wherein the alkyl moieties of the aryl-(C₁-C₆) alkyl- and heteroaryl-(C₁-C₆) alkyl groups can be optionally substituted with from one to three fluoro atoms, and where the aryl and heteroaryl moieties of these groups can optionally be substituted with one or more substituents, preferably from zero to two substutuents, independently selected from halo, oxo, nitro, amino, cyano, (C₁-C₆) alkyl optionally substituted with from one to three fluorine atoms and (C₁-C₆) alkoxy optionally substituted with from one to three fluorine atoms; and wherein one of the substituents on ring Q can be an alkyl chain that forms a 3 to 6 membered spirocyclic ring with a carbon atom of ring Q that is not adjacent to a heteroatom of ring Q; with the proviso that there can not be more than one oxo substituent on ring Q and there can not be more than one spirocyclic alkyl substituent on ring Q;
or a pharmaceutically acceptable salt thereof.

2. A compound of the formula 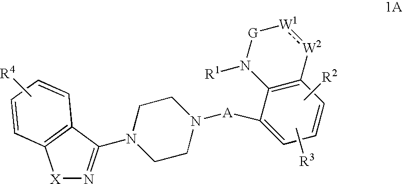
wherein X is sulfur, SO, SO₂, oxygen, or NR;
R is hydrogen, (C₁-C₆) alkyl optionally substituted with from one to three fluorine atoms, (C₁-C₆) alkoxy optionally substituted with from one to three fluorine atoms, aryl, —C(O)(C₁-C₃) alkyl, or —C(O)(C₁-C₃) alkoxy;
A is —(CH₂)ₙCH₂—, wherein n is an integer from one to four, wherein one of the CH₂ groups that is not adjacent to the piperazine nitrogen can optionally be replaced by an oxygen atom;
R¹, R⁵, R⁶, R⁷, and R⁸ are independently selected from hydrogen, (C₁-C₆) alkyl optionally substituted with from one to three fluorine atoms, (C₁-C₆) alkoxy optionally substituted with from one to three fluorine atoms, aryl, aryl-(C₁-C₆) alkyl-, (C₁-C₆) alkenyl optionally substituted with from one to three fluorine atoms, heteroaryl and heteroaryl-(C₁-C₆) alkyl-, wherein the alkyl moieties of the aryl-(C₁-C₆) alkyl- and heteroaryl-(C₁-C₆) alkyl groups can be optionally substituted with from one to three fluoro atoms, and where the aryl and heteroaryl moieties of these groups can optionally be substituented with one or more substituents, preferably from zero to two substutuents, independently selected from halo, nitro, amino, cyano, (C₁-C₆) alkyl optionally substituted with from one to three fluorine atoms and (C₁-C₆) alkoxy optionally substituted with from one to three fluorine atoms;
or R¹ is ZR⁹ wherein Z is —C(O)—, —C(O)O—, —C(O)NH—, —S(O)₂— or —S(O)₂NR¹⁰, wherein the hyphen to the left of each of the foregoing moieties represents the bond to NR¹ in structural formula 1A, and the hyphen to the right of each of the foregoing moieties represents the bond to R⁹ in structural formula 1A;
R², R³ and R⁴ are independently selected from hydrogen, (C₁-C₆) alkyl optionally substituted with from one to three fluorine atoms, (C₁-C₆) alkoxy optionally substituted with from one to three fluorine atoms, hydroxy, halogen, nitro, cyano, amino, (C₁-C₆) alkylamino and di-(C₁-C₆) alkylamino;
G is —C(═O)— or CH₂;
W¹ is C(R⁵)(R⁶), CHN(R⁵)(R⁶), CHC(═O)NR⁵R⁶ or C(OH)(R⁵);
W² is C(R⁷)(R⁸), CHN(R⁷)(R⁸), CHC(═O)NR⁵R or C(OH)(R⁷);
the broken line extending from W¹ to W² represents an optional double bond;
or one of R⁵, R⁶, R⁷, and R⁸, if present, that is attached to a carbon atom, can form, together with the carbon to which it is attached and together with another of R⁵, R⁶, R⁷, and R⁸ that is present and attached to a carbon or nitrogen atom, and the carbon or nitrogen atom to which it is attached, a three to seven membered saturated or unsaturated carbocyclic or heterocyclic ring; and
with the proviso that when there is a double bond between W¹ and W², then: (a) if W¹ is C(R⁵)(R⁶), either R⁵ or R⁶ is absent; and (b) if W¹ is CHN(R⁵)(R⁶), either the H atom attached to the ring carbon or R⁵ or R⁶ is absent; and (c) if W¹ is C(OH)(R⁵), either the OH group attached to the ring carbon or R⁵ is absent; (d) if W¹ is CHC(═O)NR⁵R⁶, either the hydrogen attached to the ring carbon or C(═O)NR⁵R⁶ is absent; (e) if W² is C(R⁷)(R⁸), either R⁷ or R⁸ is absent; (f) if W² is CHN(R⁷)(R⁸), either the H atom or R⁷ or R⁸ is absent; (g) if W² is C(OH)(R⁷), either the OH group or R⁷ is absent; and (h) if W¹ is CHC(═O)NR⁷R⁸, either the hydrogen attached to the ring carbon or C(═O)NR⁷R⁸ is absent;
and the pharmaceutically acceptable salts of such compounds.

3. A compound according to claim 1 that is selected from the following compounds and their pharmaceutically acceptable salts:
8-[2-(4-1,2-Benzisothiazol-3-yl-piperazin-1-yl)-ethyl]-4,4-dimethyl-3,4-dihydro-1H-quinolin-2-one;
8-[2-(4-1,2-Benzisothiazol-3-yl-piperazin-1-yl)-ethyl]-1,4,4-trimethyl-3,4-dihydro-1H-quinolin-2-one;
8-[2-(4-1,2-Benzisothiazol-3-yl-piperazin-1-yl)-ethyl]-6-fluoro-4,4-dimethyl-3,4-dihydro-1H-quinolin-2-one;
8-[2-(4-1,2-Benzisothiazol-3-yl-piperazin-1-yl)-ethyl]-3,4-dihydro-1H-quinolin-2-one hydrochloride;
8-[2-(4-1,2-Benzisothiazol-3-yl-piperazin-1-yl)-ethyl]-3,3-dimethyl-3,4-dihydro-1H-quinolin-2-one;
8-[2-(4-1,2-Benzisothiazol-3-yl-piperazin-1-yl)-ethyl]-4-phenyl-3,4-dihydro-1H-quinolin-2-one;
8-[2-(4-1,2-Benzisothiazol-3-yl-piperazin-1-yl)-ethyl]-3-methyl-1H-quinolin-2-one;
8-[2-(4-1,2-Benzisothiazol-3-yl-piperazin-1-yl)-ethyl]-3,4-dimethyl-1H-quinolin-2-one;
8-[2-(4-1,2-Benzisothiazol-3-yl-piperazin-1-yl)-ethyl]-4-methyl-1H-quinolin-2-one;
8-[2-(4-1,2-Benzisothiazol-3-yl-piperazin-1-yl)-ethyl]-4-phenyl-1H-quinolin-2-one;
8-[2-(4-1,2-Benzisothiazol-3-yl-piperazin-1-yl)-ethyl]-4-triflouromethyl-1H-quinolin-2-one;
8-[2-(4-1,2-Benzisothiazol-3-yl-piperazin-1-yl)-ethyl]-4,4,5-methyl-3,4-dihydro-1H-quinolin-2-one;
8-[2-(4-1,2-Benzisothiazol-3-yl-piperazin-1-yl)-ethyl]-6-chloro-4,4-dimethyl-3,4-dihydro-1H-quinolin-2-one;
8-[3-(4-1,2-Benzisothiazol-3-yl-piperazin-1-yl)-propyl]-6-chloro-4,4-dimethyl-3,4-dihydro-1H-quinolin-2-one;
8-[2-(4-1,2-Benzisothiazol-3-yl-piperazin-1-yl)-ethyl]-4-isopropyl-1H-quinolin-2-one;
8-[2-(4-1,2-Benzisothiazol-3-yl-piperazin-1-yl)-ethyl]-4-ethyl-1H-quinolin-2-one;
8-[2-(4-1,2-Benzisothiazol-3-yl-piperazin-1-yl)-ethyl]-4-propyl-1H-quinolin-2-one; and
8-[2-(4-1,2-Benzisothiazol-3-yl-piperazin-1-yl)-ethyl]-3-ethyl-4-methyl-1H-quinolin-2-one.

4. A compound according to claim 2 wherein A is an alkylene group and n is one.

5. A compound according to claim 2 wherein R⁴ is hydrogen and one or both of R² and R³ are hydrogen.

6. A compound according to claim 2 wherein R¹, R⁵, R⁶, R⁷ and R⁸ are selected, independently, from hydrogen and (C₁-C₃)alkyl.

7. A compound according to claim 2, wherein one or both of R² and R³ are hydrogen.

8. A pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delerium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising an amount of a compound according to claim 1, or a pharmaceutically acceptable salt thereof, that is effective in treating such disorder or condition, and a pharmaceutically acceptable carrier.

9. A method for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delerium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to a mammal in need of such treatment an amount of a compound according to claim 1, or a pharmaceutically acceptable salt thereof, that is effective in treating such disorder or condition.

10. A method according to claim 9, wherein the disorder or condition that is being treated is selected from major depression, single episode depression, recurrent depression, child abuse induced depression, postpartum depression, dysthymia, cyclothymia and bipolar disorder.

11. A method according to claim 9, wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

12. A method according to claim 9, wherein the disorder or condition that is being treated is selected from autism, pervasive development disorder, and attention deficit hyperactivity disorder.

13. A method according to claim 9, wherein the disorder or condition that is being treated is selected from generalized anxiety disorder, panic disorder, obsessive-compulsive disorder, post-traumatic stress disorder, and phobias, including social phobia, agoraphobia, and specific phobias.

14. A method according to claim 9, wherein the disorder or condition being treated is schizophrenia with concomitant depression.

15. A method according to claim 9, wherein the disorder or condition being treated is schizophrenia with concomitant anxiety.

16. A method of treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delerium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal, including a human, comprising administering to said mammal:
(a) a compound of the formula 1 or a pharmaceutically acceptable salt thereof; and
(b) another pharmaceutically active compound that is an antidepressant or an anti-anxiety agent, or a pharmaceutically acceptable salt thereof;
wherein the active agents “a” and “b” are present in amounts that render the combination effective in treating such disorder or condition.

17. A pharmaceutical composition for treating a disorder or condition selected from single episodic or recurrent major depressive disorders, dysthymic disorders, depressive neurosis and neurotic depression, melancholic depression including anorexia, weight loss, insomnia, early morning waking or psychomotor retardation; a typical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior behavioral disturbances associated with mental retardation, autistic disorder, and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delerium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy in a mammal comprising:
(a) a compound according to claim 1 or a pharmaceutically acceptable salt thereof;
(b) another pharmaceutically active agent that is an antidepressant or an anti-anxiety agent; and
(c) a pharmaceutically acceptable carrier.

18. A method according to claim 16, wherein the disorder or condition being treated is schizophrenia.

19. A method according to claim 16, wherein the disorder or condition that is being treated is selected from schizophrenia, schizoaffective disorder, delusional disorder, substance-induced psychotic disorder, brief psychotic disorder, shared psychotic disorder, psychotic disorder due to a general medical condition, and schizophreniform disorder.

20. A method according to claim 16, wherein the disorder or condition that is being treated is selected from autism, pervasive development disorder, and attention deficit hyperactivity disorder.

21. A method according to claim 16, wherein the disorder or condition that is being treated is selected from generalized anxiety disorder, panic disorder, obsessive-compulsive disorder, post-traumatic stress disorder, and phobias, including social phobia, agoraphobia, and specific phobias.

22. A method according to claim 16, wherein the compound of formula 1 is administered to a human for the treatment of any two or more comorbid disorders or conditions selected from those disorders and conditions referred to in any of the above methods.

23. A method according to claim 16, wherein the disorder or condition being treated is schizophrenia with concomitant depression.

24. A method according to claim 16, wherein the disorder or condition being treated is schizophrenia with concomitant anxiety.
